(19) United States
(12) Patent Application Publication
(10) Pub. No.: US 2025/0327083 A1
(43) Pub. Date: Oct. 23, 2025

(54) Nucleic acid scaffolded artificial immune complexes

(71) Applicant: THE GOVERNING COUNCIL OF THE UNIVERSITY OF TORONTO, Toronto (CA)

(72) Inventors: Leo CHOU, Toronto (CA); Travis DOUGLAS, Toronto (CA)

(73) Assignee: THE GOVERNING COUNCIL OF THE UNIVERSITY OF TORONTO, Toronto, ON (CA)

(21) Appl. No.: 19/184,931

(22) Filed: Apr. 21, 2025

Related U.S. Application Data
(60) Provisional application No. 63/636,744, filed on Apr. 20, 2024.

(30) Foreign Application Priority Data
Apr. 25, 2024 (CA) 3236444
May 8, 2024 (LU) LU507169

(51) Int. Cl. C12N 15/117; C07K 16/44

(52) U.S. Cl. CPC C12N 15/117; C07K 16/44; C07K 2317/33; C07K 2317/35; C07K 2317/52; C07K 2317/92; C07K 2318/00; C12N 2310/17

(57) ABSTRACT

An artificial immune complex (IC) free in solution, the artificial IC comprising a nucleic acid (NA) folding comprising stapled NA strands, the NA folding having an outer surface patterned with addressable sites and epitopes bound to the addressable sites and displayed in three dimensions for recruiting antibodies free in solution.

### CROSS REFERENCE TO RELATED APPLICATIONS

This application claims the benefit under 35 U.S.C. 119 of: (i) U.S. Provisional Ser. No. 63/636,744, filed Apr. 20, 2024, (ii) Canadian Patent Application No. 3,236,444, filed Apr. 25, 2024, and (iii) Luxembourg Patent Application No. LU507169, May 8, 2024, the contents of each of which are hereby incorporated by reference into the present disclosure.

### FIELD OF TECHNOLOGY

The present disclosure relates to nucleic acid foldings and to nucleic acid scaffolded artificial immune complexes.

### BACKGROUND INFORMATION

Immune complexes (ICs) are antigen-antibody assemblies that form in response to humoral immunity against antigens. IC formation plays a critical role in the elimination of pathogens, both by directly blocking and interfering with their functions, and by engaging Fc receptors (FcRs) on immune cells to initiate effector functions. Many immune cells, including neutrophils, monocytes, macrophages, dendritic cells, and B cells, express one or more FcRs, including a diverse repertoire of inhibitory and activating subtypes. By signaling through FcRs, ICs trigger a range of effector responses such as complement activation, cellular phagocytosis, cytokine secretion, and antigen presentation. Beyond their role in natural immunity, these effector responses also modulate the efficacy of therapeutic antibodies, immune-modulating drugs, and vaccines. On the other hand, mis-regulation of these effector responses is known to cause damage to the body. For example, the binding of antibodies to self-antigens expressed in host tissues can lead to the local accumulation of insoluble ICs, which triggers inflammation, tissue damage, and autoimmune diseases such as lupus¹.

Physical characteristics of ICs such as size, geometry, composition, and valency regulate their effector responses via modulating FcR binding and clustering, cell uptake, and intracellular processing. For example, antigens arrayed on microbes permit the binding of multiple antibodies to induce multivalent FcR crosslinking for efficient immune cell clearance. On the other hand, various antigens and their spatial organizations cause larger IC formation to be impermissible, thus reducing their clearance and prolonging their persistence in circulation. Various mathematical and experimental models have been developed to study multivalent IC binding with Fc receptors and predict their effector outcomes²⁻⁶. Additionally, multimeric but not monomeric ICs are trafficked to lysosomes following uptake by immune cells such as macrophages and dendritic cells. These interactions have direct consequences on the cellular immune response directed against the contents contained within the IC. For example, antigen uptake via the Fc-FcR pathway has been shown to promote antigen presentation on major histocompatibility complex (MHC) molecules, leading to enhanced T cell priming. On the other hand, self-DNA, which is normally non-immunogenic, has been shown to activate Toll-like receptor 9 when complexed with antibodies, and these auto-reactive ICs have been isolated from patients with autoimmune disorders. Thus, IC formation modulates both the innate and adaptive immune responses towards the contents associated within the IC, and engineering such IC structure and composition allows control over such responses.

Current methods of synthesizing ICs lack precise control over their structure and composition. The most common approach, complexation, involves simply admixing the antigen with either a polyclonal antibody mixture, or two or more monoclonal antibodies that bind separate epitopes on the antigen. This method typically results in the assembly of heterogeneous aggregates whose average sizes are roughly adjusted based on the antibody-antigen stoichiometry. Recombinant expression involves fusing the target antigen to the C-terminus of the heavy chain of the antibody that binds the antigen. Co-expression of this fusion protein, along with the light chain of the antibody, results in a self-reactive antibody that spontaneously assembles into ICs^{7,8}. These two methods also lack the ability to package payloads within the IC for cell delivery. Finally, antigens have been arrayed in multivalent fashion on nano- and micro-particles for assembly with antibodies into ICs. However, it remains difficult to prescribe the antigen valency and spacing on these materials for controlling the overall shape and structure of the assembled ICs.

### SUMMARY OF DISCLOSURE

In one embodiment, the present disclosure provides for an artificial immune complex (IC) free in solution, the artificial IC comprising a nucleic acid (NA) folding comprising stapled NA strands, the NA folding having a surface patterned with addressable sites and epitopes bound to the addressable sites and displayed in three dimensions for recruiting antibodies free in solution.

In one embodiment of the artificial IC of the present disclosure, the artificial IC further comprises the antibodies scaffolded to the NA folding such that the fragment antigen-binding (Fab) region of the antibodies free in solution are bound to one or more of the epitopes patterned on the NA folding and the Fc portion of the antibodies orients away from the surface of the NA folding, wherein the patterning of the epitopes promotes an immune response against the entire artificial IC.

In another embodiment of the artificial IC of the present disclosure, the addressable sites comprise single-stranded NA handles (handles) patterned on the surface of the NA folding, each handle having an end attached to the outer surface of the NA folding, and wherein each epitope includes a single-stranded NA sequence that hybridizes with the handles.

In another embodiment of the artificial IC of the present disclosure, the addressable sites comprise functional groups and each epitope is bound directly to one functional group on the NA foldings. In one aspect, functional groups include amines, carboxylic acids, alcohols, aldehydes, esters, thiols, azides, alkynes, dibenzocyclooctyne, tetrazines, trans-cyclooctene, modified nucleotides, nucleosides, phosphoroamidites, or enzymatic labeling via sortags, snap-tags, clip-tags, spacers such as 1-20 carbons, ethylene glycols, and so forth.

In another embodiment of the artificial IC of the present disclosure, the epitopes bound to the addressable sites are patterned in regular geometric groupings on the surface of the NA folding.

In another embodiment of the artificial IC of the present disclosure, the epitopes bound to the addressable sites are patterned in pairs, or in clusters of 3 or more copies of epitopes.

In another embodiment of the artificial IC of the present disclosure, the geometric groupings include squares, rectangles, equilateral triangles, isosceles triangles, cylinders, diamonds, rhomboids, and polyhedrons.

In another embodiment of the artificial IC of the present disclosure, the NA folding is a 3-dimensional (3D) NA folding, and the regular geometric groupings and epitopes within the regular geometric groupings are radially, axially and azimuthally spaced on the surface of the 3D NA foldings to control antibody binding, structure and/or composition of the artificial IC.

In another embodiment of the artificial IC of the present disclosure, the artificial IC comprises a single NA folding in which the epitopes are arranged on the surface in clusters of 2 or more epitopes per cluster, and wherein epitopes within each cluster are spaced apart on the surface of the NA foldings at a distance within a binding tolerance of the antibodies free in solution, and spacing between neighboring clusters of epitopes is outside the binding tolerance of the antibodies free in solution, thereby preventing the antibodies free in solution from binding neighboring clusters of epitopes and from crosslinking epitopes on separate NA foldings.

In another embodiment of the artificial IC of the present disclosure, the artificial IC comprises an assembly of multiple NA foldings crosslinked via the fragment antigen-binding (Fab) region of the antibodies, wherein all epitopes patterned on the surface of the NA folding are spaced apart at a distance outside a binding tolerance of the antibodies free in solution, thereby promoting the antibodies free in solution crosslinking between two or more artificial ICs.

In another embodiment of the artificial IC of the present disclosure, the artificial IC comprises a mixture of single NA foldings with an assembly of multiple NA foldings, wherein the epitopes are patterned on the surface of each NA folding in clusters of two or more epitopes, and wherein at least one cluster includes at least two epitopes spaced apart at a distance within a binding tolerance of the antibodies free in solution, and at least one cluster includes at least two epitopes spaced apart at a distance that is greater than the binding tolerance of the antibodies free in solution, thereby controlling the number of cross linking antibodies and the overall number of NA foldings in the assembly.

In another embodiment of the artificial IC of the present disclosure, the surface of the NA folding is coated with a lysine multimer having a PEG moiety conjugated to the backbone of the lysine multimer and/or to an end of the lysine multimer, and wherein the lysine multimer contains at least 20 lysine units.

In another embodiment, of the artificial IC of the present disclosure, the surface of the NA folding is coated with a lysine multimer having a PEG moiety conjugated to the backbone of the lysine multimer and/or to an end of the lysine multimer, and wherein the lysine multimer contains between 2 and 16 lysine units.

In another embodiment of the artificial IC of the present disclosure, each NA folding is patterned with a total of 3 copies of epitopes. In another embodiment of the artificial IC of the present disclosure, each NA folding is patterned with a total of 6 copies of epitopes. In another embodiment of the artificial IC of the present disclosure, each NA folding is patterned with a total of 9 copies of epitopes. In another embodiment of the artificial IC of the present disclosure, each NA folding is patterned with a total of 12 copies of epitopes. In another embodiment of the artificial IC of the present disclosure, each NA folding is patterned with a total of 16 copies epitopes. In another embodiment of the artificial IC of the present disclosure, each NA folding is patterned with a total of 18 copies epitopes.

In another embodiment of the artificial IC of the present disclosure, the artificial IC comprises an assembly of multiple NA foldings crosslinked via the fragment antigen-binding (Fab) region of the antibodies.

In another embodiment of the artificial IC of the present disclosure, the artificial IC comprises a single NA folding (“monomeric artificial IC”).

In another embodiment of the artificial IC of the present disclosure, the antibodies include IgG, IgM, IgA, IgD, IgE and/or immunoglobulin fragments.

In another embodiment of the artificial IC of the present disclosure, the nucleic acid of the NA foldings is DNA, RNA or DNA/RNA hybrid.

In another embodiment of the artificial IC of the present disclosure, the NA includes chemically modified bases.

In another embodiment of the artificial IC of the present disclosure, the NA folding includes nucleic acid sequences encoding for therapeutic or immunomodulatory proteins. In one embodiment the NA sequence is a DNA molecule.

In another embodiment of the artificial IC of the present disclosure, the NA folding carries a cargo or combination of cargos.

In another embodiment of the artificial IC of the present disclosure, the cargo is incorporated into an inner lumen of the NA folding or to an outer surface of the NA folding.

In another embodiment of the artificial IC of the present disclosure, the cargo is bound to the addressable sites (i.e., to the handles or to the functional groups) or bound directly to the surface of the NA folding.

In another embodiment of the artificial IC of the present disclosure, the cargo includes an RNA molecule, a small molecule, a macromolecule, an adjuvant peptide, a protein, a chemotherapeutic, and/or an immune-modulatory drug, including any combinations thereof. In one aspect, the RNA molecule is one or more of mRNA, miRNA, siRNA, InRNA and so forth.

In another embodiment of the artificial IC of the present disclosure, each NA folding is in the form of a barrel, a rod, a sphere or a polyhedron.

In another embodiment, the present disclosure provides for a method of manufacturing a synthetic or artificial (in this document the terms “synthetic” and “artificial” are used interchangeably) immune-complex (IC), the method comprising: (a) mixing in an aqueous solution (i) nucleic acid (NA) foldings and epitopes, each NA folding having a surface patterned with addressable sites to bind the epitopes in the solution, or (ii) NA foldings and staple NA strands conjugated with epitopes, thereby obtaining a mixture of epitopes bound to the addressable sites of NA foldings; and (b) adding antibodies to the mixture of epitopes bound to the NA foldings, wherein the antigen-binding portion of the antibodies binds to the epitopes bound to the surface of NA foldings, such that the Fc portion of the antibodies orients away from the surface of the NA folding.

In one embodiment of the method of manufacturing an artificial IC of the present disclosure, each addressable site comprises a functional group attached to staple NA strands at specific sites on the surface of the NA folding, and wherein each functional group is an incorporation site for the epitopes.

In another embodiment of the method of manufacturing an artificial IC of the present disclosure, each addressable site comprises single-stranded NA handles (handles) patterned at specific sites on the surface of the NA folding, each handle having an end attached to the surface of the NA folding and a free end that orients away from the surface of the NA folding, and each epitopes having a single stranded anti-handle NA sequence, and wherein step (a) includes mixing in the aqueous solution the NA foldings including the handles with the epitopes having the anti-handle NA sequence under conditions favorable for the hybridization of the handles to the anti-handles.

In another embodiment of the method of manufacturing an artificial IC of the present disclosure, the epitopes are bound to the addressable sites in clusters of two or more epitopes per cluster, and wherein epitopes within a cluster are space apart at a distance within a binding tolerance of the antibodies free in solution, and spacing between neighboring clusters of epitopes is outside the binding tolerance of the antibodies free in solution thereby preventing the antibodies free in solution from crosslinking epitopes in neighboring pairs within one NA folding and from crosslinking epitopes on separate NA foldings.

In another embodiment of the method of manufacturing an artificial IC of the present disclosure, all epitopes patterned on the surface of the NA folding are spaced apart at a distance that is outside a binding tolerance of the antibodies free in solution, thereby promoting the antibodies free in solution crosslinking between two or more artificial ICs.

In another embodiment of the method of manufacturing an artificial IC of the present disclosure, the epitopes are bound to the addressable sites in clusters of two or more epitopes per cluster, and wherein each includes (i) epitopes spaced apart within a binding tolerance of the antibodies free in solution, and (ii) epitopes spaced apart on the surface of the NA folding at a distance outside the binding tolerance of the antibodies free in solution, thereby promoting mixture of antibodies free in solution to both cross linking between two or more NA foldings and to binding epitopes within a cluster on a single NA folding.

In one embodiment of the method of manufacturing an artificial IC of the present disclosure, the method further comprises coating the surface of the NA folding with a lysine multimer having a PEG moiety conjugated to the backbone of the lysine multimer and/or to an end of the lysine multimer, and wherein the lysine multimer contains at least 20 lysine units.

In another embodiment of the method of manufacturing an artificial IC of the present disclosure, each addressable site comprises a functional group attached to staple NA strands at specific sites on the surface of the NA folding, and wherein each functional group is an incorporation site for the epitopes.

In another embodiment of the method of manufacturing an artificial IC of the present disclosure, each addressable site comprises single-stranded NA handles (handles) patterned at specific sites on the surface of the NA folding, each handle having an end attached to the surface of the NA folding and a free end that orients away from the surface of the NA folding, and each epitopes having a single stranded anti-handle NA sequence, and wherein step (a) includes mixing in the aqueous solution the NA foldings including the handles with the epitopes having the anti-handle NA sequence under conditions favorable for the hybridization of the handles to the anti-handles.

In another embodiment of the method of manufacturing an artificial IC of the present disclosure, the epitopes are bound to the addressable sites in clusters of two or more epitopes per cluster, and wherein epitopes within a cluster are space apart at a distance within a binding tolerance of the antibodies free in solution, and spacing between neighboring clusters of epitopes is outside the binding tolerance of the antibodies free in solution thereby preventing the antibodies free in solution from crosslinking epitopes in neighboring pairs within one NA folding and from crosslinking epitopes on separate NA foldings.

In another embodiment of the method of manufacturing an artificial IC of the present disclosure, all epitopes patterned on the surface of the NA folding are spaced apart at a distance that is outside a binding tolerance of the antibodies free in solution, thereby promoting the antibodies free in solution crosslinking between two or more artificial ICs.

In another embodiment of the method of manufacturing an artificial IC of the present disclosure, the epitopes are bound to the addressable sites in clusters of two or more epitopes per cluster, and wherein each includes (i) epitopes spaced apart within a binding tolerance of the antibodies free in solution, and (ii) epitopes spaced apart on the surface of the NA folding at a distance outside the binding tolerance of the antibodies free in solution, thereby promoting mixture of antibodies free in solution to both cross linking between two or more NA foldings and to binding epitopes within a cluster on a single NA folding.

In another embodiment, the present disclosure provides for a method of delivering a cargo (including combination of cargos) to a target site in a subject, the method comprising administering to the subject the artificial IC of the present disclosure carrying the cargo. In aspects, the target site includes lymph nodes, spleen, tonsils, and/or diseased tissue. In one aspect the disease tissue includes a tumor and/or cancerous tissue.

In one embodiment of the method of delivering a cargo to a target site in a subject of the present disclosure, the artificial IC is a multimeric artificial IC comprising an assembly of multiple NA foldings crosslinked via the fragment antigen-binding (Fab) region of the antibodies, or the artificial IC is a monomeric artificial IC comprising a single NA folding.

In another embodiment, the present disclosure provides for a method of inducing an immune response in a subject, the method comprising administering to the subject the artificial IC according to an embodiment of the present disclosure.

In one embodiment of the method of inducing an immune response in a subject, the artificial IC is a multimeric artificial IC comprising an assembly of multiple NA foldings crosslinked via the fragment antigen-binding (Fab) region of the antibodies, or the artificial IC is a monomeric artificial IC comprising a single NA folding.

In another embodiment, the present disclosure relates to the use of the nucleic acid folding of the present disclosure for therapeutic applications and/or for research.

### BRIEF DESCRIPTION OF THE DRAWINGS

The following figures illustrate various aspects and preferred and alternative embodiments.

FIGS. 1A-1B—Overview of immune complex (IC) formation scaffolded by DNA origami. 1A: A 30×60 nm DNA barrel serves as the backbone of the IC to scaffold ssDNA handles in varying spatial arrangements. ssDNA anti-handles with a 3′ biotin are functionalized site specifically to the surface of the barrel. Anti-biotin IgG2a antibodies are incubated with biotinylated barrels to form ICs. 1B: Theoretical spacing of functionalization sites along with antibody binding confirmations to barrel surface antigens.

FIGS. 2A-2D—Antigen valency and antibody-antigen stoichiometry determines IC dispersity. 2A, 2D abstraction of the outer surface of the DNA origami barrel with the prescribed antigen spacings. 2B, AGE gels of the respective barrel designs incubated with increasing antibody titers ranging from 0 (no antibody) to 10 (10:1 antibody-to-antigen ratio). 2C and 2D, Transmission electron micrographs of the respective barrel-ICs at a 1:1 (c) and 4:1 antibody-to-antigen ratio (d). Insets depict single unbound antibody (row 2, columns 2) and antibodies bridging multiple barrels (row 2, columns 4). Scale bars, 50 nm.

FIGS. 3A-3D—Programmed antigen spacings exploit the binding tolerance of IgG antibodies to control IC crosslinking and size. 3A: Schematic of theoretical antibody binding events dictated by antigen spacing on the outer surface of the barrel. 3B: 2D abstractions of antigen spacings on the outer surface of the DNA origami barrel. 3C: AGE of the respective barrel designs incubated with increasing antibody titers ranging from 0 (no antibody) to 10 (10:1 antibody-to-antigen ratio). 3D: TEMs of the respective DNB-ICs at a 1:1 antibody-to-antigen ratio. Inset (micrograph in column 3) shows a portion of the IC aggregate with negative stain (Scale bars, 50 nm in the inset and columns 1, 2; 200 nm in column 3).

FIGS. 4A-4F—DNA origami-scaffolded immune complexes bind Fc-gamma receptors on macrophages. 4A: Schematic of the experimental method is shown, where DNB-ICs dye labeled with AF546 were incubated with RAW264.7 macrophages for 1 hour at 4C. Samples were then imaged using confocal microscopy. 4B: 2D abstraction of antigen spacings on the outer surface of the barrel accompanied with 6-sites for AF546 dye labeling. 4C: Cell binding assay of uncoated DNB-ICs to RAW264.7 macrophages (scale bars, 5 μm). Single z-slice confocal images of macrophage binding by uncoated, AF546- labeled DNB-ICs and corresponding antibody-negative controls. Cells were incubated with 0.2 nM of DNA origami for 1 hr at 4° C. 4D, 4F: Dot-plot quantifications for the mean AF546 fluorescence signal per cell. Each grey dot represents the mean fluorescence intensity of one cell. (*p≤0.05, **p≤0.01, ***p<0.001, or ****p≤0.0001). Each black dot represents the mean intensity of one biological replicate. For each condition, N=3 biological replicates were performed, and image analysis was performed on >250 cells; ns, not significant. The means of multiple groups were compared using a one-way ANOVA, post-hoc Tukey's test. Error bars represent the standard deviation. 4E: Cell binding assay of K₁₀-PEG₅ₖ coated DNB-ICs to RAW264.7 macrophages (scale bars, 5 μm). Single z-slice confocal images of macrophage binding by uncoated, AF546- labeled DNB-ICs and corresponding antibody-negative controls. Cells were incubated with 0.2 nM of DNA origami for 1 hr at 4° C.

FIGS. 5A-5L—DNA origami immune complexes engage FcγRs to alter the rate and magnitude of uptake in a cell-type and shape dependent manner. 5A: Methodology for FcγR colocalization assay. Cell binding for 1 hour at 4° C. followed by uptake at 37° C. for 30 minutes. Samples were labelled with AF647 anti-FcγR either following binding or following uptake to observe co-localization with AF546 labeled barrels. AF647 anti-FcγRI antibody binds FcγRI (neg., negative control, iso., isotype control). Multi-channel single-z confocal images are shown. Scale bars, 5 μm. 5B: Kinetics of DNB-IC uptake by RAW 264.7 macrophages. Cells were incubated with 1 nM of DNA origami barrel and corresponding ICs at 37° C. for the indicated times. Dot-plot quantifications for the mean AF647 signal per cell. DNB-ICs were visualized using origamiFISH (Wang et al. Nature Nanotechnology, 2024). Each colored dot represents mean fluorescence intensity of one cell. Each black dot represents mean fluorescence intensity for one field of view (*p≤0.05, **p≤0.01, ***p≤50.001, or ****p≤0.0001) and N=3 biological replicates; ns, not significant (3 fields of view per biological replicate, ≥28 cells per field of view for a total sample size >250 cells). The means of multiple groups were compared using a one-way ANOVA, post-hoc Dunnett's test. Error bars represent the standard deviation. b, representative single z-slice confocal images are shown. Scale bars, 10 μm. Measured uptake (30 min exposure) of monomeric barrel-ICs in 5C, RAW264.7 macrophages or 5E, DC2.4 dendritic cells, analyzed by flow cytometry. Bar plots of median fluorescence intensity (N=3) (*p≤0.05, **p≤0.01, ***p≤0.001, or ****p≤0.0001); ns, not significant. The means of multiple groups were compared using a one-way ANOVA, post-hoc Tukey's test. 5D and 5F: Confocal microscopy images of the respective samples (Max z-projection of 2 z-slices) detecting the uptake of barrel-ICs with origami-FISH. 5G: Diagram of the 400×7 nm rods (6-helix bundle), with regularly spaced antigens separated by 14 nm. Designs have either 6, 12, or 16 biotins. 5H: TEMs of rod-ICs (arrows indicate IgG antibodies, scale bar—100 nm). 51: AGE gel of the biotinylated rod incubated with increasing antibody titers ranging from 1:1 to 4:1 (antibody to antigen ratio). 5J, 5K: Median fluorescence intensity of either RAW264.7s (5J) or DC2.4s (5K) exposed to rod-ICs and rod-ICs with increasing titers of antibody analyzed by flow cytometry. (*p≤0.05, **p≤0.01, ***p≤50.001, or ****p≤0.0001) and N=3 biological replicates; ns, not significant. The means of multiple groups were compared using a one-way ANOVA, post-hoc Tukey's test. 5L: max z projection of representative confocal images detecting the uptake of rod-ICs using origami-FISH.

FIGS. 6A-6F—Controlling IC size to increase uptake into macrophages and dendritic cells. 6A: 2D abstraction of antigen spacings on the outer surface of the barrel for multimeric IC assembly. 6B: AGE gels of the respective barrel designs incubated with increasing antibody titers ranging from 0 (no antibody) to 2 (2:1 antibody to antigen ratio). 6C: Transmission electron micrographs of the respective multimeric DNB-ICs at a 1:1 antibody to antigen ratio (samples from columns left to right; multimeric DNB03-IC, DNB06-IC, DNB12-IC, and DNB18-IC), Scale bars, 100 nm. 6D: ImageJ was used to create masks to measure the size (nm²) of particles that make up the various designs from captured TEM images. At least 200 particles were measured for each design. 6E: The measured sizes of the particles were plotted on a histogram according to their frequency in each field of view multiplied by their surface area to accordingly weight and account for the degree of crosslinking that occurs between designs to control IC size. The plot shows the non-linear fit of the surface areas of the measured particles weighted by their frequency (log₁₀ area, nm²). For this plot, the design, the weighted means of the particle area (log₁₀ area, nm²), and R² goodness of fit, are as follows; DNB (Ab-), 3.722, 0.9934, DNB03-IC, 3.598, 0.8885, DNB06-IC, 6.077, 0.8485, DNB12-IC, 6.747, 0.9386, and DNB18-IC, 6.162, 0.6841. TEM visualization of the resulting ICs demonstrated an increase in IC surface area (i.e. size) from 3.9×103 nm², 1.2×10⁶ nm², to 5.6×10⁶ nm² for multimeric designs with 3, 6, and 12 antigens, respectively. 6F: Uptake of the various sized multimeric DNB-IC designs, in RAW264.7 macrophages or DC2.4 dendritic cells, analyzed by flow cytometry. To evaluate uptake, 1 nM of each respective multimeric DNB-IC design or a respective control were exposed to either RAW264.7 macrophages or DC2.4 dendritic cells for 30 mins.

FIG. 7—Agarose gel electrophoresis demonstrating incorporation and purification of biotinylated DNA origami barrels. S, p7308 ssDNA scaffold; C, crude folded DNA origami with 10× excess staple strands; successful folding is demonstrated by a gel mobility shift from the ˜7.5 kb ssDNA scaffold. S1, supernatant from the first round of PEG purification; R1, DNA origami barrels after the first round of PEG purification; BB, biotinylated DNA origami barrel; S2, supernatant from the second round of PEG purification; PBB is the purified biotinylated barrel with a distinct band shift and removal of excess staple strands.

FIG. 8—ICs were co-labeled with an AF647 dye-labeled secondary antibody specific for the primary anti-biotin IgG2a to co-label the ICs. S, is the ssDNA; C is the folded crude sample folded with 10× excess of staple strands. Samples were first incubated with increasing ratios of primary (1°) anti-biotin IgG2a to form ICs. IC samples were then co-incubated with secondary (2°) AF647 dye labeled anti-IgG2a. DNB (−) indicates sample is without DNA barrels.

FIGS. 9A-9B—Agarose gel electrophoresis of increasing antigen valency designs (DNB00, 06, 09, 12, and 18). 9A, 9B: Agarose gel electrophoresis of ICs formulated with increasing number of antigens, i.e. 0, 6, 9, 12, and 18, according to the spatial pattern shown in FIG. 2A. ICs showed increasing band shifts with increasing antigen valency. Sample stability and storage was assessed on agarose gel electrophoresis after 120 hours of storage at 4° C. in 1× folding buffer.

FIG. 10—Formulation concentration impacts IC dispersity. Gel mobility shift assay on agarose gel electrophoresis; samples DNB06 (top) and DNB18 (bottom) were incubated with IgG antibodies at a fixed 1:1 antibody-to-antigen ratio at DNA origami concentrations ranging from 2 to 40 nM. L, DNA ladder; S, is the ssDNA, DNB is the respective biotinylated DNA origami barrel without IgG. DNB06-ICs appear largely monomeric across the range of concentration tested, whereas DNB18-ICs appears increasingly multimeric at 16 nM and above. Representative transmission electron microscopy (TEM) images are shown on the right. Scale bar, 250 nm.

FIG. 11—FcγRI immuno-labeling of RAW 264.7 murine macrophages. All samples were stained for 24 hours. For reliable specificity all labeled samples included in the manuscript used 0.5 μg of Fc block.

FIG. 12—Assessment of DNB-IC stability following cold storage and incubation in different media. 1×FB, 1× folding buffer; DMEM, Dulbecco's Modified Eagle Medium; FBS, fetal bovine serum. Stability was assessed for up to 4 hours, 3.5 hours longer than the cell uptake experiments performed in this study.

FIG. 13—Confirmation of AF546-labeled DNA origami barrels by agarose gel electrophoresis. Successful dye labeling was observed via colocalization of AF546 signal with DNA origami band on the gel; successful IC formation and coating were confirmed by gel shift in samples following addition of antibody and oligolysine-PEG, respectively. Antibody:antigen ratios were 1:1 across all designs, same as used in cell experiments.

FIG. 14—Characterizing the morphology of RAW264.7s following exposure to DNB-ICs. Cells were pre-treated with IFN-gamma for 16 hours. Media was replaced with fresh media and left to equilibrate for 2 hours prior to cell uptake. 1 nM of K₁₀-PEG₅ₖ coated DNB-ICs or respective controls were introduced to RAW264.7s cells for 8, 16, and 24 hours. At 8- and 16-hours uptake media was removed and replaced with fresh media. For all samples brightfield microscopy images were acquired to assess morphological changes at the designated exposure times and following media replacement up to the 24-hour mark.

FIG. 15—Characterizing the cytokine expression of RAW264.7s following exposure to DNB-ICs. Cells were pre-treated with IFN-gamma for 16 hours. Media was replaced with fresh media and left to equilibrate for 2 hours prior to cell uptake. 1 nM of K₁₀-PEG₅ₖ coated DNB-ICs or respective controls were introduced to RAW264.7s cells for 8, 16, and 24 hours. ELISAs were performed for IFN-beta and TNF-alpha. MC, media control, Ab12+, 12 nM of free IgG2a, DNB00, Ab6+, barrels co-incubated with 6 nM of free IgG2a, DNB00, Ab12+, barrels co-incubated with 12 nM of free IgG2a, agg=IC aggregate or multimeric IC.

FIG. 16—Quantifying the number of antibodies per structure on monodisperse DNB-ICs. DNB-IC formulated at the 1:1 antigen-to-antibody ratio (the condition used in all cell experiments) were purified by PEG precipitation to remove any unbound IgG. Replicate samples were resuspended in 1× folding buffer and fluorescence signals at AF546 (barrel) and AF647 (IgG) were measured using a plate reader. Raw fluorescence values were converted to concentration based on standard curves. The average number of antibodies per DNA origami was determined by dividing the concentration of the antibody by the concentration of DNA origami. DNB06-IC and DNB12-IC had an average of 4 and 8 antibodies per DNA origami, respectively. ****p≤0.0001, N=3 replicates. The means of multiple groups were compared using a one-way ANOVA, post-hoc Tukey's test. Error bars represent the standard deviation. Agarose gel electrophoresis of AF546-labeled DNB-ICs formulated with AF647-labeled anti-biotin IgG2a, confirming colocalization of the antibody to the DNA origami barrel. Different lanes contain different antibody-to-antigen ratios ranging from 0 to 4.

FIG. 17—Graphs illustrating proposed applications for nucleic acid scaffolded artificial immune complex drug-carriers.

FIGS. 18A-18B—Graphs illustrating non-exhaustive examples of designs or patterns on a 2-dimensional (2D) surface (18A) and a 3-dimensional (3D) surface (18B).

FIG. 19—IL-6 secretion of RAW 264.7 macrophages in response to DNA scaffolded immune complexes according to one embodiment of the present disclosure. Respective samples were exposed to RAW 264.7 macrophages for 8 hours (1 nM of DNA barrels). Negative control is media only. IgG2a, cells were exposed to 12 nM of free IgG2a (equimolar amount to [1:1] antibody-antigen samples). DNB00, +IgG2a indicates DNA barrels without antigen co-incubated with free IgG2a. DNB12-IC, IgG2a indicates monomeric DNA barrel immune complexes with 12 antigens. Brackets indicate [antibody:antigen] ratio, 1 nM of monomeric DNB12-IC at [1:1] has 12 nM of IgG2a.

FIG. 20—TNF-α secretion of IFN-γ stimulated RAW 264.7 macrophages in response to DNA scaffolded immune complexes according to one embodiment of the present disclosure. Respective samples were exposed to IFN-γ stimulated RAW 264.7 macrophages for 8, 16, and 24 hours (1 nM of DNA barrels).

FIG. 21—Agarose gel electrophoresis demonstrating direct incorporation of biotinylated staples, purification, and opsonization of DNA barrels. S, p7308 ssDNA scaffold; C, crude folded DNA origami with 6× excess staple strands. Successful folding is demonstrated by a gel mobility shift from the ˜7.5 kb ssDNA scaffold. DNA origami barrels after one round of PEG purification. Ab-, indicates no IgG2a antibody. Ab+, indicates incubated with IgG2a antibody. Antigen patterning determines either monomeric IC-barrels or multimeric IC-barrels.

FIG. 22—DNA barrels folded with AF750 dye directly incorporated into the inner lumen of the barrel. 12 and 12×; DNA barrels with 12 biotins spaced according to the monomeric design (12) or the multimeric design (12×), respectively.

FIG. 23—AF750 dye labelled barrel immune complexes (dye directly incorporated to the inner lumen of the barrel. Design was iterated from previous designs with tracking dye functionalized to the outer lumen using handle-anti-handle conjugation.

FIGS. 24A to 24F—Conjugation of tracking fluorophore to the inner lumen improves signal detection in tissues over time. 24A: 7-10-week-old female C57BL/6 mice were intramuscularly injected in the right caudal thigh with 100 nM of the respective DNA nanostructure formulations. At 2-, 8-, and 24-hours mice were sacrificed, and lymph nodes and tissues were harvested to be imaged on IVIS. B-F, Biodistribution of AF750® dye-labelled DNB00 co-incubated with free IgG2a—either with the AF750® dye incorporated directly to the scaffold on the inner lumen or conjugated to the outer surface via handle-anti-handle chemistry and PBS control measured across various tissues over time. 24B-24F: (24B) lymph nodes (6 lymph nodes: proximal and distal—iliac, inguinal, and popliteal), (24C) injection site, (24D) liver, (24E) kidneys, and (24F) spleen. Data are presented as mean±SEM. Reference for each curve in FIGS. 24B-24F: 1: PBS control; 2: Handle anti-handle AF750 tracking dye; 3: Inner lumen AF750 tracking dye.

FIG. 25—DNA barrel scaffolded ICs co-functionalized with OVA antigen. S3, Supernatant of 3rd round of purification showing unbound excess OVA. OVA-DNB12, DNA barrel with 12 biotin antigens and OVA conjugated to its outer surface. OVA-DNB12 was incubated with increasing concentrations of anti-biotin IgG2a antibody, titrated relative to the number of biotin moieties on the DNA barrel. The indicated molar ratios (0, 0.5, 1, 2, 4) represent IgG2a to antigen. Immune complex (OVA-DNB12-IC) formation was assessed by agarose gel electrophoresis, where a band shift indicates successful binding of IgG2a to the biotinylated DNA scaffold bearing OVA.

FIG. 26—Uptake of DNA barrel scaffolded ICs co-functionalized with OVA antigen in RAW264.7s. Scale bars, 10 μm.

FIGS. 27A to 27F—Monomeric immune complexes reduce variability in lymph node trafficking while maintaining consistent and comparable biodistribution over time. 27A: 7-10-week-old female C57BL/6 mice were intramuscularly injected in the right caudal thigh with 100 nM of the respective DNA nanostructure formulations. At 2-, 8-, and 24-hours mice were sacrificed, and lymph nodes and tissues were harvested to be imaged on IVIS. 27B-27F: Biodistribution of AF750® dye-labelled DNB00 co-incubated with equimolar amounts of free IgG2a, DNB12, monomeric DNB12-ICs, multimeric DNB12-ICs and PBS control measured across various tissues over time. 27B-27F: (27B) lymph nodes (6 lymph nodes: proximal and distal—iliac, inguinal, and popliteal), (27C) injection site, (27D) liver, (27E) kidneys, and (27F) spleen. Data are presented as mean±SEM.

FIG. 28—Biodistribution of DNA scaffolded immune complexes.

FIG. 29—Immune complex dispersity controls lymph node penetration and distribution. Scale bars, 100 μm.

FIG. 30—Immune complex dispersity controls lymph node penetration and distribution. Scale bars, widefield 100 μm and zoomed insets 20 μm.

### DETAILED DISCLOSURE

### Definitions

As used in the specification and claims, the singular form “a,” “an” and “the” include plural references unless the context clearly dictates otherwise. For example, the term “a cell” includes a plurality of cells, including mixtures thereof.

As used herein, the terms “comprising,” “including,” “having” are intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this disclosure. Embodiments defined by each of these transition terms are within the scope of this disclosure.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above.

All numerical designations, e.g., pH, temperature, time, concentration, and molecular weight, including ranges, are approximations which are varied (+) or (−) by increments of 0.1 or 1.0 as is appropriate. It is to be understood, although not always explicitly stated that all numerical designations are preceded by the term “about” which includes a standard deviation of about 15%, or alternatively about 10% or alternatively about 5%. It also is to be understood, although not always explicitly stated, that the reagents described herein are merely exemplary and that equivalents of such are known in the art.

Nucleic acid origami is the nanoscale folding of nucleic acid to create arbitrary two- and three-dimensional shapes at the nanoscale. The process of producing nucleic acid origami involves the folding of a long scaffolding single strand of nucleic acid, such as viral DNA (typically the 7,249 bp genomic DNA of M13 bacteriophage) aided by multiple smaller “staple” strands. These shorter staple strands bind the longer scaffolding strand in various places, resulting in the formation of a pre-defined two- or three-dimensional shape. CAD software is used to assist in creating the predefined shape of the nucleic acid origami (caDNAno, vHelix). Examples of shapes include a smiley face and a coarse map of China and the Americas, along with many three-dimensional structures such as cubes, rods, barrels and so forth. In this document the terms “nucleic acid origami,” “nucleic acid folding,” “nucleic acid nanostructure,” “particle(s)” are used interchangeably to refer to nucleic acid origami.

The term “nucleic acid”, as used in this document, relates to a nucleotide sequence, such as ribonucleic acid (RNA), deoxyribonucleic acid (DNA), or a mixture (hybrid) of RNA and DNA.

“Immunoglobulin” or “antibody” or “antibody fragment” are used interchangeably to refer to an antigen-binding peptide produced by plasma cells or produced artificially. In one embodiment, the antibodies useful for the present disclosure include IgG (including IgG1, IgG2, IgG3, IgG4), IgA (including IgA1, IgA2), IgM, IgD and IgE. Other useful antibodies include hybrids of natural antibody isotypes, chimeric antibodies, humanized antibodies, and antibody fragments such as a Fab fragment, a F(ab′)2 fragment, a diabody, a single chain Fv fragment, a tetrabody, a triabody, a disulfide bond-stabilized Fv or a heavy chain VHH fragment. In another embodiment, the antibodies include polyclonal antibodies and/or monoclonal antibodies. In another embodiment, useful antigen-binding peptide include peptides that are not antibodies, such as receptor proteins, small affinity ligands that are designed to mimic antibody-binding properties (Affibody®, Affilin®, Affimer®, Affitin, Alphabody), artificial proteins designed to bind antigens (Anticalin®), nanobody (antibody fragments derived from heavy-chain only IgG antibodies), designed ankyrin repeat proteins or DARPin, antibody-mimetics such as dual-affinity retargeting antibody or DART and so forth.

“Geometric pattern” or “geometric grouping” or “geometric shapes” are used interchangeably to refer to a shape or shapes formed by at minimum of two non-parallel lines connecting the antigens placed on the surface of a 2D or 3D nucleic acid origami, alternatively a geometric pattern is formed by at least three antigens placed in a non-collinear manner on the 2D or 3D surface of a nucleic acid origami. Squares, rectangles, triangles, cylinders, diamonds, rhomboids, parallelograms, polyhedrons, and shapes derived from these shapes are non-exhaustive examples of geometric groupings. For the purpose of this document (including the claims) a simple line (i.e., just one line) connecting two antigens is not considered a geometric grouping/shape/pattern.

The binding affinities of antibodies against epitopes on a surface change with the distances amongst epitopes. The term “binding tolerance” is used in this document to refer to the distance between epitopes on a nucleic acid (NA) surface that permit a single antibody to bivalently bind epitopes on the same NA surface (within the binding tolerance of the antibody). When the spacing is outside the binding tolerance, bivalent binding to epitopes on the same NA surface is restricted, permitting antibodies in solution to cross-link epitopes on separate artificial IC NA surfaces.

The artificial IC of the present disclosure are free in solution. “Free in solution” means that all facets of a 3D NA folding of the present disclosure are not attached or immobilized to a solid surface, but instead all such facets of the 3D NA folding are exposed and free to interact with other NA foldings and molecules also free in solution (such as antibodies). Examples of solutions include water, water containing organic solvents (to mediate solubilization of certain molecules), aqueous salt-containing buffers such as saline or phosphate-buffered saline, bodily fluids including saliva, tears, urine, mucus, whole blood, plasma, serum, intracellular fluids, interstitial fluids, lymphatic fluids, cerebrospinal fluid, serous fluids, and synovial fluids. In one embodiment, the water contains up to 10% organic solvents.

### Overview

The present disclosure provides for structural nucleic acid (DNA, RNA, or DNA/RNA hybrid) nanotechnology to program antigen arrays to engineer synthetic immune complexes (ICs).

### Artificial Immune Complexes

In one embodiment, the present disclosure provides for artificial immune complex (IC) comprising nucleic acid (NA) origami or foldings (in this document NA scaffold, NA origami and NA folding are used interchangeable) as scaffolds to incorporate various epitopes, including antigens to template the assembly of ICs with defined shapes and valency. NA foldings can be fabricated in custom sizes and shapes, with epitopes spatially patterned in programmed densities, stoichiometries, and spacings at the nanometre-level⁹⁻¹⁶. In one embodiment, the epitope on the NA foldings are of the same structure and recognized by the same antibody, for example, the same epitope of one origin (haptens, immunogens, native antigens, tumor antigens, autoantigens, endogenous antigens, and exogenous antigens such as a pathogen). In another embodiment, the NA foldings include epitopes having different structures such that each epitope is recognized by a different antibody, for example different epitopes of the same origin or epitopes of different origins.

As such, in one embodiment, the present disclosure provides for an artificial immune complex (IC) free in solution, the artificial IC comprising a nucleic acid (NA) folding comprising stapled NA strands, the NA folding having an outer surface patterned with addressable sites and epitopes bound to the addressable sites and displayed in three dimensions for recruiting antibodies free in solution. In one aspect, the artificial IC further comprises the antibodies scaffolded to the NA folding such that the fragment antigen-binding (Fab) region of the antibodies free in solution bind to one or more of the epitopes patterned on the NA folding and the Fc portion of the antibodies orients away from the surface of the NA folding, wherein the patterning of the epitopes promotes an immune response against the entire artificial IC.

In one embodiment, the addressable sites comprise single-stranded NA handles (handles) patterned on the outer surface of the NA folding, each handle having an end attached to the outer surface of the NA folding, and wherein each epitopes includes a single-stranded NA sequence that hybridizes with the handles.

In another embodiment, the addressable sites comprise functional groups and each epitopes is bound directly to one functional group on the NA foldings. Examples of functional groups include amines, carboxylic acids, alcohols, aldehydes, esters, thiols, azides, alkynes, dibenzocyclooctyne, tetrazines, trans-cyclooctene, modified nucleotides, nucleosides, phosphoroamidites, or enzymatic labeling via sortags, snap-tags, clip-tags, spacers such as 1-20 carbons, ethylene glycols, and so forth.

The structural features of nucleic acid (DNA, RNA, or DNA/RNA hybrid) foldings determine antibody-epitope interactions and IC formation in solution. In one embodiment, epitope spacing matching the spatial tolerance of immunoglobulin G (IgG) is used to determine artificial IC size and dispersity. The physical structure of the NA folding-assembled ICs of the present disclosure influence their uptake by FcγR-expressing immune cells including macrophages and dendritic cells.

The present disclosure highlights artificial IC comprising nucleic acid (DNA, RNA, or DNA/RNA hybrid) foldings as a novel and inventive platform to probe the influence of artificial IC structure on antibody-mediated immune cell interactions and effector responses. The platform of the present disclosure can be extended to study the impact of other IC design parameters, such as antibody and FcR subtypes, on immune response to ICs. Since nucleic acid origamis of the present disclosure can additionally be functionalized with other payloads such as passenger antigens and immune-modulatory drugs, the artificial ICs of the present disclosure can be used as platforms to build IC-based therapeutics and vaccines.

Although the examples provided below use DNA as the structural backbone, other nucleic-acid assemblies such as RNA assemblies, DNA/RNA hybrid assemblies, can also be used. The use of nucleic acid structural backbones allows for tuning of IC solubility, size, shape, and antibody valency. The scaffolds of the present disclosure can display epitopes in arbitrary or non-arbitrary spatial patterns, valences, stoichiometries, and nanoscale spacings. The prescribed antigen patterns scaffolded by nucleic acids of the present disclosure allow to control how antibodies bind to form synthetic, soluble in aqueous solutions, immune complexes. The strategy implemented by the present disclosure is compatible with antibodies of any antigen specificity, provided the antigen can be displayed on the scaffold (antigens can be freely soluble peptides, proteins, sugars, or nucleic acids). Additionally, the scaffold of the present disclosure can be engineered in arbitrary 1 D, 2D, and 3D shapes to alter the overall IC geometry. Physical features of immune complexes, such as solubility, size, shape, and antibody valency dictate their delivery, binding, uptake, and intracellular processing by immune cells and tissues.

As such, in one embodiment, the present disclosure provides for an artificial IC free in solution comprising a nucleic acid folding or nucleic acid origami, patterned with copies of epitopes. The epitopes may be distributed anywhere on an outer surface of the NA folding and/or an inner surface of the NA folding. In one embodiment, the copies of the epitopes are arranged in geometric groupings on a surface (outer and/or inner) of the nucleic acid folding. The geometric groupings of the epitopes include, for example, arrangements such as squares, rectangles, triangles, cylinders, diamonds, rhomboids, parallelograms, polyhedrons, and geometric groupings that derived from these non-exhaustive examples. In one embodiment, the nucleic acid origami is patterned with repeating multiples of the antigen.

FIG. 18 illustrates non-exhaustive examples of geometric groupings of the epitopes portrayed in 2D (FIG. 18A) and shown on the outer surface of the 3D NA foldings (FIG. 18B). In the 3D NA folding, the epitopes within the geometric groupings are radially, axially and azimuthally spaced on the surface of the 3D NA foldings to control antibody binding and overall structure and/or composition of the artificial IC. As further described below, the geometric groupings and the epitopes within the geometric groupings are spaced to control the binding tolerance of the antibodies against the copies of the epitopes to produce assemblies of monomeric NA foldings ICs, nanoscale ICs or aggregates of NA foldings.

Epitopes include full antigens, haptens, immunogens, native antigens, tumor antigens, autoantigens, endogenous antigens, and exogenous antigens (food allergen, pollen, aerosols, etc.).

In one embodiment, the nucleic acid origami of the present disclosure is soluble in aqueous solution.

The structure of the nucleic acid origami of the present disclosure can be tailored to desired properties.

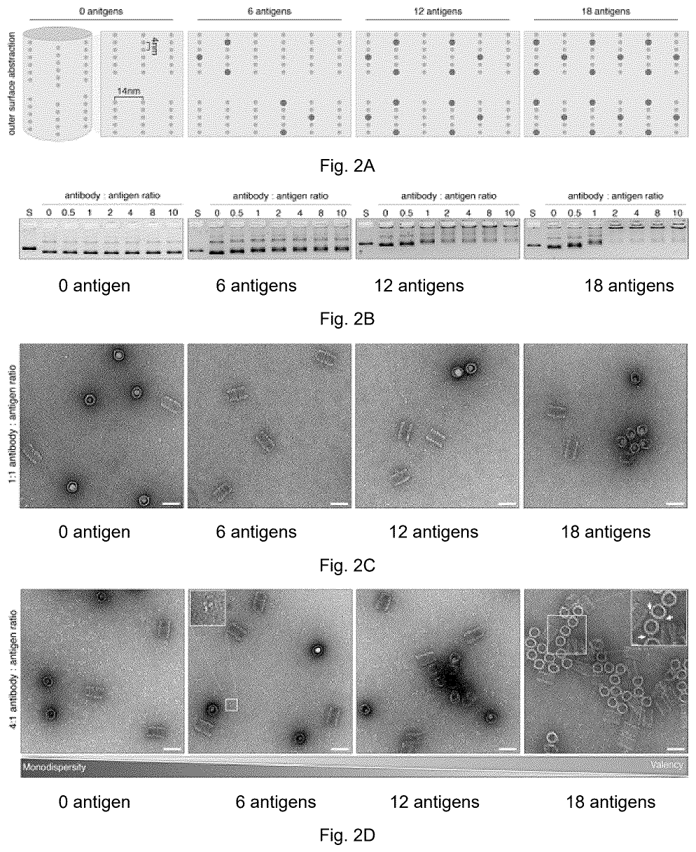

In one embodiment, increasing antigen valency on nucleic acid origami/folding correlated with increased antibody binding (FIG. 9). In another embodiment, increasing the antigen valency and the antibody to antigen ratio promote the assembly of nucleic acid foldings into larger, crosslinked ICs (FIGS. 2B and 2C), while reduced at reduced antigen valency the nucleic acid foldings remain monodisperse (FIGS. 2B and 2C). As such, in another embodiment, the present disclosure provides for multiple nucleic acid origami assembled via the fragment antigen-binding (Fab) region of the antibodies/immunoglobulins attached to the antigens on the surface of the nucleic acid origami (see the transmission electron micrographs of FIG. 2C).

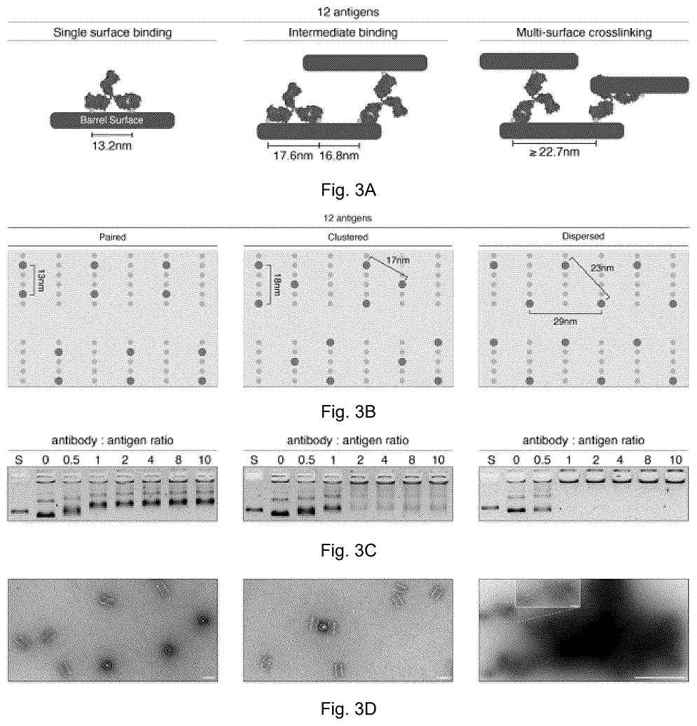

With reference to FIGS. 3A-3D, in another embodiment, epitope spacing within the nucleic acid foldings of the present disclosure is used to control antibody binding. In one embodiment, copies of the epitopes are spaced apart on the nucleic acid folding at a distance matching or within a binding tolerance of antibodies against the copies of the epitopes. In this embodiment, the nucleic acid folding induces bivalent binding of a single antibody between neighboring pairs of epitopes on a single NA folding particle surface while preventing antibody binding across epitopes on separate NA foldings (FIG. 3A, left). In another embodiment, copies of the antigen are spaced apart on the surface of the nucleic acid foldings at a distance outside the binding tolerance of immunoglobulins against the copies of the epitopes. In this embodiment, the nucleic acid foldings of the artificial IC induces monovalent antibody binding and promote the antibodies crosslinking epitopes on separate NA foldings (FIG. 3A, right). In another embodiment, copies of the antigen are spaced apart on the surface of the nucleic acid origami at a distance permissive to both bivalent and monovalent antibody binding (FIG. 3A, middle).

The defined epitope spacings determine the structure, composition and/or function of the artificial ICs of the present disclosure. In one embodiment, the NA foldings of the artificial ICs of the present disclosure take a 3D form. As such, in one embodiment, the geometric groupings and the epitopes are radially, axially and azimuthally spaced on the surface of the 3D NA foldings to control antibody binding and overall structure and/or composition of the artificial IC.

In another embodiment of the artificial IC of the present disclosure, the surface of the NA folding is coated with a lysine multimer having a polyethylene glycol (PEG) moiety conjugated to the backbone of the lysine multimer and/or to an end of the lysine multimer. In one embodiment the lysine multimer contains at least 20 lysine units. In another embodiment, the lysine multimer contains between 20 and 40 lysine units. In another embodiment, between 2 to 16 of the lysine multimer is conjugated to the PEG moiety.

In another embodiment, the present disclosure provides for a method of reducing cross-linking among antibodies on a nucleic acid folding, the method comprising, or consisting essentially of, or consisting of providing a nucleic acid folding having copies of an antigen of the immunoglobulins spaced apart at a distance that matches a binding tolerance of the immunoglobulins.

In another embodiment, the present disclosure provides for a method of increasing cross-linking among immunoglobulins on a nucleic acid origami/folding, the method comprising, or consisting essentially of, or consisting of providing a nucleic acid folding having copies of epitopes of the antibodies spaced apart at a distance that is larger than a binding tolerance of the antibodies.

### Ic Binding and Uptake by FcγR-Expressing Immune Cells

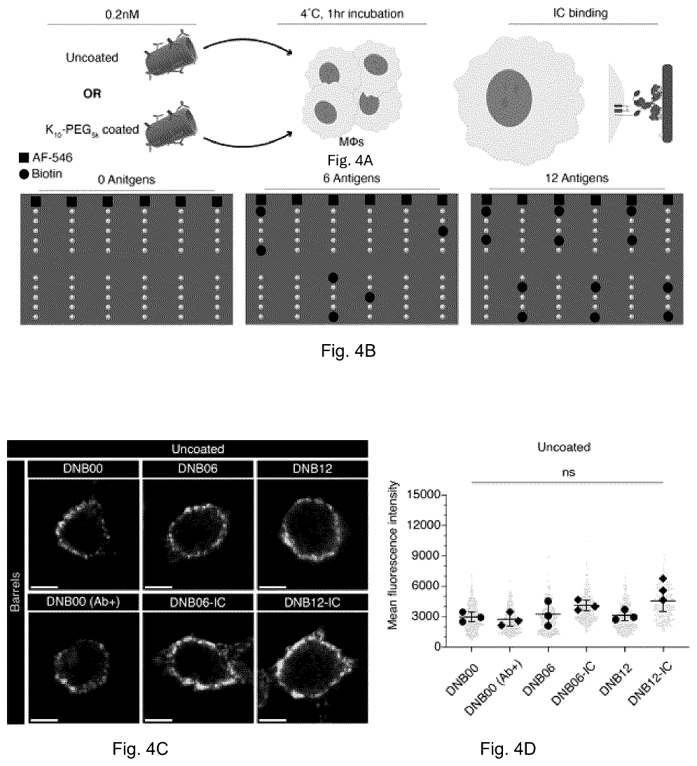

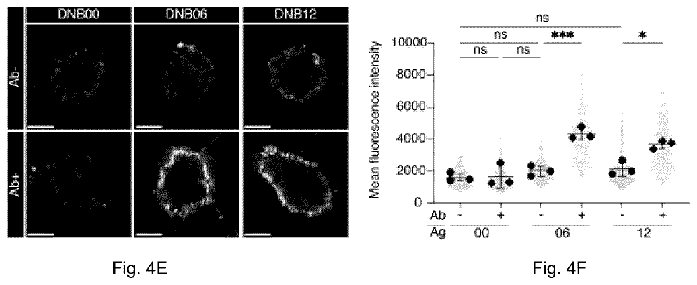

The ICs of the present disclosure are shown to bind and internalize by FcγR-expressing immune cells, such as macrophages and dendritic cells (see FIG. 4).

### Applications

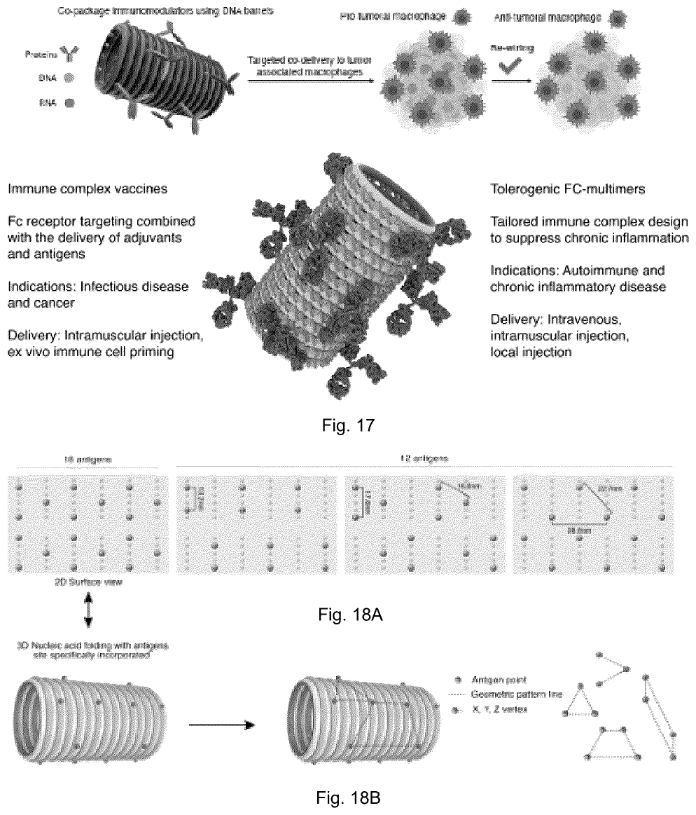

In embodiments, the artificial IC of the present disclosure itself can be therapeutic, such as the NA folding containing sequences that encode for therapeutic proteins or have immuno-modulatory effects such as binding to nucleic-acid sensors in cells (see FIG. 17). These sequences can be chemically modified to further dampen or enhance the therapeutic effects. In embodiments, the nucleic acid scaffold of the artificial IC of the present disclosure can additionally be chemically functionalized with one or a combination of payloads or cargos, which can be small molecules or macromolecules, including RNA molecule (mRNA, miRNA, siRNA, InRNA and so forth) adjuvants, peptides, proteins, chemotherapeutics, and/or immune-modulatory drugs. In one embodiment, each cargo is bound to the addressable sites on the NA foldings or directly to the surface of the NA foldings. In embodiments, the cargo is bound to the NA folding via covalent and noncovalent bonds. Noncovalent bonds include hydrogen, electrostatics, ionic, hydrophobic, affinity interactions and so forth. Covalent bonds include chemical conjugation and enzymatic conjugation chemistries. This allows the co-delivery of multiple drugs along with the antigen to the target immune cells or tissues by the IC to modulate immune response to the IC. The functionalization chemistries can encompass covalent coupling/electrostatic binding/hydrophobic interactions/nucleic-acid hybridization or any other non-covalent molecular self-assembly techniques. The nucleic acid scaffolded immune complex nano-carriers of the present disclosure also include ICs formed with either full antibodies or any partial form of an antibody's Fc domain (ex. recombinantly expressed Fc domains) via any coupling/conjugation/hybridization strategies which include but are not limited to incorporating the whole or parts of an antibody to the nucleic acid scaffold via antibody fused DNA-DNA hybridization, ligand-ligand chemistries, click chemistries, peptide couplings, esterification, amidation, etc., to form a complex with the scaffold surface. With reference to FIG. 17, the nucleic acid scaffolded immune complexes of the present disclosure have diverse applications: (1) In embodiments, the nucleic acid scaffolded immune complexes of the present disclosure can be used as a research tool to probe Fcy-receptor biology on mammalian cells expressing any Fcy receptor. The platform can be used to characterize various antibody-receptor interactions which include but are not limited to: Fcy receptor binding affinity, Fcy receptor-mediated phagocytosis of ICs, overall IC uptake, rate of IC uptake, antigen presentation, cell polarization, efficiency of nucleic acid scaffolded immune complex carrier cargo delivery as facilitated by antigen valency, spacing, stoichiometry, and patterning. (2) In embodiments, the nucleic acid scaffolded immune complexes of the present disclosure can have applications as a therapeutic delivery vehicle to deliver immune therapeutics to immune cells that express Fc receptors, which include macrophages, dendritic cells, neutrophils, monocytes, NK cells, and B cells. The nucleic acid scaffolded immune complex nano-carriers complexes of the present disclosure can be co-functionalized with any combination of small molecule or macromolecular drugs and co-deliver them to these cells. (3) In embodiments, the nucleic acid scaffolded immune complex nano-carriers complexes of the present disclosure can have applications as vaccines by enhancing immunogenicity to the antigens. Binding of Fc to activating Fc receptors enhances antigen immunogenicity, which can be further enhanced by co-delivering immune stimulating payloads (DNA, RNA, or other vaccine adjuvants). (4) In embodiments, the nucleic acid scaffolded immune complex nano-carriers complexes of the present disclosure can be engineered to treat auto-immune diseases by interacting with inhibitory Fc receptors using specific antibody isotypes, and by co-delivering immune-suppressive drugs within the IC. (5) In embodiments, the nucleic acid scaffolded immune complex nano-carriers complexes of the present disclosure can be used as boosters for infectious disease where patterned antigen display can be leveraged to induce in situ IC formation to enhance uptake and immune response. (6) In embodiments, the nucleic acid scaffolded immune complex nano-carriers complexes of the present disclosure can be used ex-vivo to deliver neoantigens to antigen presenting cells for the purpose of priming T-cells.

As such, in another embodiment, the present disclosure provides for a use of the artificial ICs of the present disclosure in the treatment of autoimmune diseases, in the treatment of infectious diseases by using antigens taken an infectious organism, as immunotherapy of cancer.

The artificial ICs of the present disclosure can be administered or delivered to a subject through any suitable form of administration or delivery, including, for example, intravenously (IV), intramuscularly (IM), subcutaneously (SQ) administration, intraperitoneally, eye drops and/or orally.

In order to aid in the understanding and preparation of the present disclosure, the following illustrative, non-limiting examples are provided.

### EXAMPLES

### Example 1

### Methods

Dna Origami Synthesis and Purification DNA barrels and rods were folded with a Thermo Fisher ProFlex PCR system according to published protocols²⁵. All ssDNA staple strands were ordered from IDT. Following thermal ramps samples were held short term at 20° C. and long term at 4° C. until purified.

### Barrel

Structures were thermally annealed using 10 nM of p7308 ssDNA scaffold (Guild Biosciences, D441-020-1 mL100) and 10-fold excess ssDNA staple strands in 1× folding buffer composed of 10 mM MgCl2, 5 mM Tris and 1 mM EDTA at pH 8. Thermal ramp cycle based on published work²⁵.

### Rod

Structures were thermally annealed using 10 nM of p7308 ssDNA scaffold in 10-fold excess ssDNA staple strands in 1× folding buffer composed of 8 mM MgCl2, 5 mM Tris and 1 mM EDTA at pH 8. Thermal ramp cycle based on prior work²⁵.

DNA Origami Purification by PEG Precipitation Samples were incubated 1:1 v/v with purification solution composed of 14% PEG 8000 (FisherScientific, BP233), 1× Tris-EDTA, 250 mM NaCl and DNA origami-specific concentrations of MgCl2 for 30 minutes at RT. Samples were then spun at 16,000 g for 40 minutes at 25° C. Supernatant was removed and samples resuspended in their respective 1× folding buffer.

### Antigen and Fluorophore Functionalization

ssDNA staples with a 21-nt handle sequence were incorporated into DNA origamis at specific sites. All functional oligonucleotides were purchased from IDT. For functionalization, anti-handle sequences containing either 3′ biotin modification and/or Alexa Fluor®546 (AF546) modification were incubated in 3-fold excess to the number of handles at RT for 1 hour. Excess anti-handles were purified with one additional round of PEG purification.

### IgG Decoration

Anti-biotin (clone 1 D4-C5) IgG2a (BioLegend, #409002) was co-incubated with the biotinylated DNA origami nanostructures at various stoichiometries for 1 hour at RT. Opsonization was characterized with gel mobility shift assays and transmission electron micrographs.

### Gel Mobility Shift Assays (GMSA)

To assess immune complex formation, GMSAs were performed and analyzed by agarose gel electrophoresis (2% agarose, 0.5×TBE buffer with 10 mM MgCl2, pre-stained with SYBR Safe (Invitrogen, S33102), 65V for 150 min at 4° C.). All gels images were captured on iBright FL1500 Imaging System (Invitrogen, A44241).

### Antibody Valency Assay

Primary anti-biotin IgG2a was labeled with Alexa Fluor®647 (AF647) using NHS ester chemistry according to manufacturer's instructions (Invitrogen, A20186). Following labeling and purification, AF546 labeled DNBs were incubated with AF647 labeled anti-biotin IgG2a. Functionalization of the AF647-labeled antibody to AF546-labeled DNBs was confirmed by signal colocalization on GMSA. Samples were then purified via PEG purification. The fluorescence signals of the resuspended samples were measured on a fluorescence plate reader (Synergy H1 Microplate reader, Biotek Instruments, 804100) and the absolute concentrations of the antibodies and DNBs were determined from respective Ab and DNB standard curves.

### Oligolysine-Poly(Ethylene Glycol) Coating

Structures were coated at 2:1 or 1:1 N:P ratios with K10-PEG5k (Alamanda Polymers, #050-KC010) for 30 mins at RT, according to published methods²³.

### Transmission Electron Microscopy

4 μL of sample was deposited on plasma treated Ted Pella formvar carbon grids (SFR, #01754-F) for 2.5 mins and then wicked away. Sample was then washed with 4 μL of 1× folding buffer. Sample was stained with 2% uranyl formate for 30 seconds and then wicked away. Sample was let dry for 5 minutes prior to imaging. Images were taken on a Talos L120C transmission electron microscope at various magnifications of 11000×, 28000×, 57000× and 92000×.

### Coverslip Preparations

Prior to cell uptake 12 mm round coverslips (Fisher Scientific, #1254581) were pre-treated with 0.1 mg/mL of Poly-D-lysine (ThermoFisher, #A3890401). Following treatment coated coverslips were washed 3-times with 1×PBS and left to dry. Coverslips were stored in ddH₂O for up to 2 weeks before use.

### Cell Binding Assay

RAW264.7 cells were seeded on treated coverslips in a 24-well plate at a density of 100,000 cells per well, 18-24 hours before the experiment. Samples were diluted to 0.2 nM in a final volume of 200 μL of Dulbecco's modified Eagle's medium (Wisent Biproducts, 319-007-CL), supplemented with 10% fetal bovine serum (Invitrogen, 12484028) and 1% penicillin-streptomycin (Sigma Aldrich, P4333-100ML). Binding occurred at 4° C. for 1 hour. For the cell binding and uptake experiments with FcγR co-labeling, immunohistochemistry staining followed the 4° C. or 37° C. incubation. Active cell media was removed, followed by a 1×PBS wash. Samples were then fixed with 4% PFA for 10 minutes at RT. Samples were then washed with 1×PBS with 3% BSA for 5 minutes, rocking on ice. Blocking was performed for 30 minutes rocking on ice (100 μLs of blocking solution per well; 1×PBS and 3% BSA with 0.5 μg of Mouse TruStain FcX). Samples were then labeled for 30 minutes rocking on ice using 0.5 μg of Alexa Fluor®546 (AF546) anti-FcγRI (anti-CD64) and then moved to 4° C. for 24 hours to continue staining.

### Cell Uptake Assay

RAW264.7 and DC2.4 cells were seeded on treated coverslips in a 24-well plate at a density of 100,000 cells per well, 18-24 hours before the experiment. IC samples were diluted either to 0.2 nM (for origamiFlSH staining and confocal microscopy) or 1 nM (for flow cytometry) with respect to the total number of DNA origami in a final volume of 200 μL Dulbecco's modified Eagle's medium (supplemented with 10% fetal bovine serum and 1% penicillin-streptomycin). These solutions were added to cells for the specified timepoints. The lower 0.2 nM concentration used for origamiFlSH confocal microscopy followed published protocols for origamiFlSH and allowed for clearer visualization and puncta analysis. The 1.0 nM concentration used in flow cytometry analysis of cell uptake was chosen based on a dose-response curve.

### Flow Cytometry

Following cell uptake, media was removed and cells were washed once with 1×PBS. After washing, 400 μL of ice-cold 1×PBS was added to each well to detach adherent cells via gentle scrapping and jetting. Cells were then fixed in 10% PFA (Fisher Scientific, 50980487) for 10 minutes at RT. Cells were then spun down for 5 minutes at 500 g and PFA was removed. Cells were then washed twice with 1×PBS by centrifugation to remove buffer and resuspended in flow cytometry staining buffer for analysis by flow cytometry (BD LSRFortessa™). A minimum of 10,000 events per sample was collected. Data was analyzed with FlowJo.

### Origami-FISH

Following uptake origami-FISH was performed according to published protocols²⁵ using split initiator probes specific to the DNA origami scaffold that served as a nucleation site for Alexa Fluor®647 labeled hairpins.

### Confocal Microscopy

Confocal images were taken on a Zeiss LSM 880 laser scanning confocal microscope (Carl Zeiss Canada) equipped with a 63× plan-apochromatic oil immersion objective (NA 1.4). Images were collected with Zen Black software (Carl Zeiss Canada). DAPI was excited with a 405 nm solid state laser and emission was collected on the PMT spectral detector from 410-585 nm. AF647 was excited with a 633 nm solid state laser and emission was collected on the PMT spectral detector from 638-755 nm. AF546 was excited with a 532 nm solid state laser and emission was collected on the PMT spectral detector from 572-620 nm. Z slices were collected over a 0.64 μm thickness over an area of 135×135 μm with an x/y pixel resolution of 0.07 μm.

### Confocal Image Quantification

Raw confocal images were processed to separate the fluorescence channel of the DNA nanostructures from the cell nuclei stain. Cell segmentation using Cellpose108 delineated individual cells and their nuclei to provide cell counts. Masks generated from segmentation defined cell boundaries to measure the mean fluorescence intensity per cell.

### TEM Size Measurements

Raw TEM images were analyzed using FIJI. A threshold was applied to micrographs to create individual masks around each particle. The surface area of each particle mask in a field of view was measured (nm²).

### Results

### Assembly of Immune Complexes (ICs) on 3D DNA Origami Scaffold

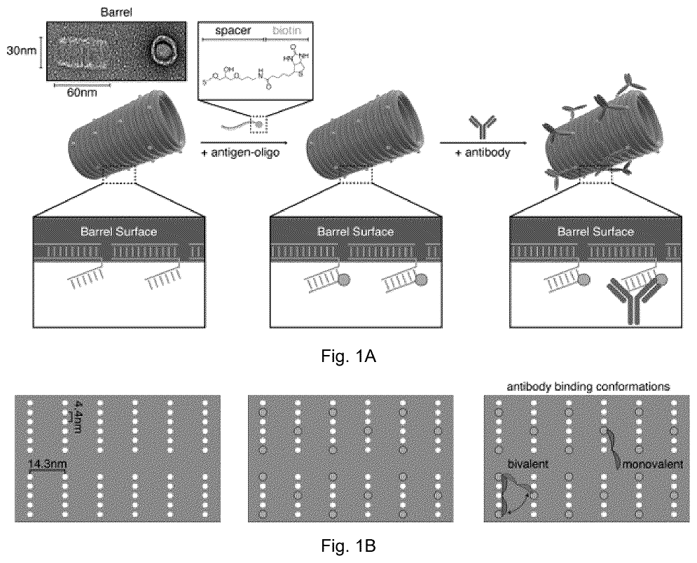

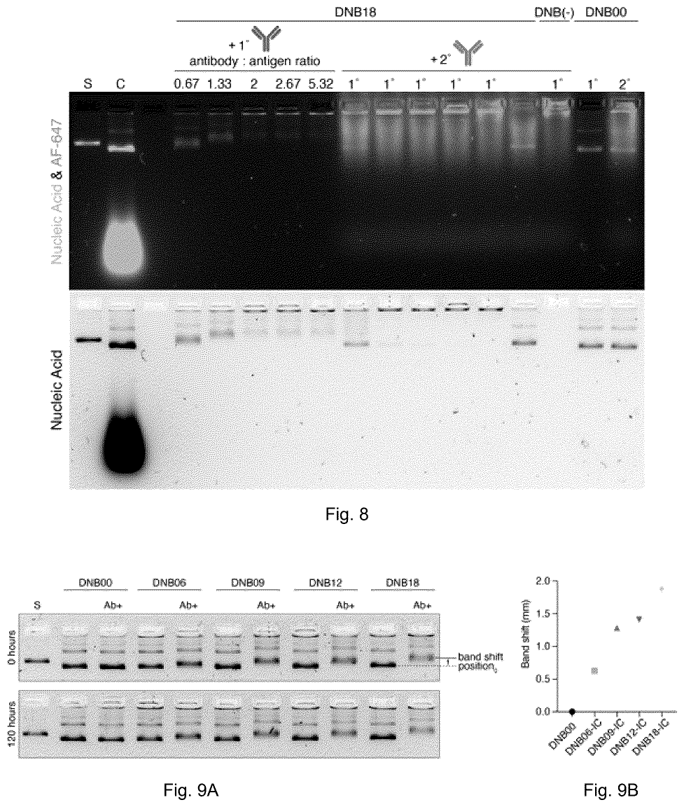

As model nanoparticle, we used a published DNA barrel (DNB)¹⁷ with dimensions of 30×60 nm (DxH) and 66 addressable sites on its outer surface (FIG. 1). DNB is a rigid 3D nanostructure that shares similar physical features such as shape, size, curvature, and aspect ratio to a viral vector¹⁸, providing relevance for understanding principles governing IC formation. As a model antigen, we patterned biotins on the DNB surface using a generalizable “handle-anti-handle” hybridization technique^{17,19}. Successful biotinylation was evidenced by a gel mobility shift assay (GMSA) (FIG. 7). To assemble ICs, biotinylated DNBs were incubated with serial dilutions of anti-biotin IgG antibody. IC formation was confirmed via GMSA by an upward band shift compared to antibody-negative controls (FIG. 8). To verify that the band shift is specific for antibody-antigen interactions, ICs were incubated with Alexa Fluor®647 (AF647) dye-labeled secondary antibodies specific for the primary IgG and re-analyzed by agarose gel electrophoresis. Here, we observed co-localization between the DNB (SYBR® Gold nucleic acid stain) and the secondary antibody (AF647, FIG. 8) only when biotinylated DNBs were mixed with the primary antibody. In contrast, biotinylated DNBs without the primary antibody did not co-localize with the secondary antibody, nor did non-biotinylated DNBs with or without the primary antibody. These results demonstrate successful IC assembly driven by specific antibody-antigen binding (FIG. 8).

### Effects of Antigen Valency, Concentration, and Antibody-Antigen Ratios on IC Size and Dispersity

After verifying the use of DNBs to assemble synthetic ICs, we next asked how different assembly conditions affected the structure of the ICs. We first tested antigen valency by creating four DNB variants, each displaying 0, 6, 12, or 18 copies of the antigen (DNB00, DNB06, DNB12, and DNB18). Each design maintained a triangular clustering of antigens spaced ˜17 nm apart (FIG. 2A). To test the effects of antibody-antigen ratios on IC assembly, we incubated each design with the anti-biotin antibody across a range of concentrations corresponding to antibody-antigen ratios ranging from 0 to 10. Analysis of the products by agarose gel electrophoresis revealed design-specific IC structures as characterized by distinct band migration patterns across the antibody-antigen ratios (FIG. 2B). In general, increasing antigen valency on DNBs correlated with increased antibody binding as evidenced by a larger gel mobility shift (FIG. 9). Interestingly, at antibody-antigen ratios of 2:1 and above, DNB18 appeared to assemble into larger, crosslinked ICs as evidenced by their inability to migrate out of the gel (FIG. 2B), while DNB06 remained monodisperse across all antibody-antigen ratios. The intermediate antigen valency, DNB12, exhibited a gel migration pattern characteristic of a mixture of crosslinked and monodisperse ICs (FIG. 2B). These effects on IC structure were driven by specific antibody-antigen interactions, as no band migration was observed for DNB00 across all antibody-antigen ratios tested. Additionally, we consistently observed binding saturation, as evidenced by a maximum gel shift, at the 1:1 antibody-antigen ratio across all designs, indicating high binding affinity between this specific antibody-antigen pair. To further verify our observations, we performed transmission electron microscopy (TEM) on ICs assembled at the 1:1 and 4:1 antibody-antigen ratios. Here, we confirmed the monodispersity of DNB06 ICs at both ratios, as well as the crosslinking observed for the DNB12 and DNB18 designs. TEM further demonstrated a difference in the average sizes of the crosslinked ICs between designs. For example, at a 4:1 antibody-antigen ratio, DNB18 formed crosslinked ICs that spanned 0.2 to 0.8 micrometers (μm) in size, compared to 0.2 to 0.4 μm for DNB12 (FIG. 2D). In the micrographs for the 4:1 antibody to antigen ratios, excess antibodies were seen scattered around the DNBs on the carbon grid. In the DNB18 micrograph these antibodies can be seen on the upright barrels, as tiny, white bulbous protrusions that formed multi-surface crosslinks with proximal barrels (column 4, row 2; white arrows).

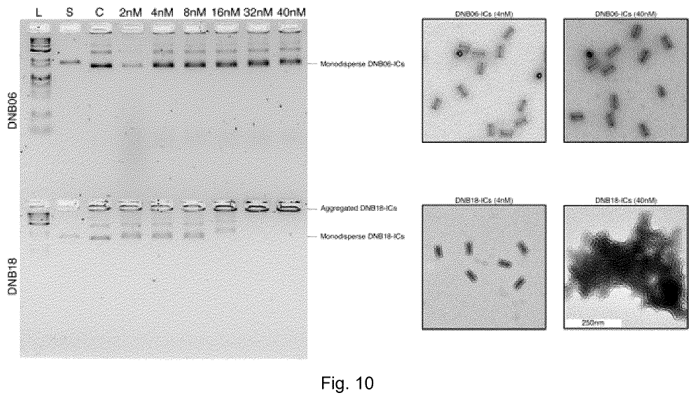

Given the effects of antibody-antigen ratio on IC size, we also asked whether the absolute concentrations of the DNB and antibody used in IC assembly influenced IC size. To this end, we incubated both DNB06 and DNB18 with antibodies at a 1:1 antibody-antigen ratio at concentrations ranging from 2 to 40 nM of the DNA origami. Here, DNB06 formed monodisperse ICs across all concentrations tested. In contrast, DNB18 induced crosslinking when assembled at concentrations of 8 nM and above (FIG. 10). These results demonstrate an interplay between the affinity of the antibody-antigen pair, antibody-antigen ratio, and nanoscale antigen pattern on IC assembly.

### Effects of Antigen Spacing on Antibody Binding Conformation and IC Dispersity

In principle, DNBs displaying 6, 12, or 18 copies of the antigen all have the capacity to assemble into large, crosslinked ICs, yet we observed design-specific crosslinking. This prompted us to ask how nanoscale antigen spacing on DNA origami controls IC formation. To investigate, we used DNB12, which assembled with antibodies into a mixed population of monodisperse and crosslinked ICs, and asked whether altering the antigen spacing on DNB12 could bias the assembly of one or the other population. Thus, we created two additional variants of DNB12 while keeping antigen valency constant. The first variant, called DNB12-bivalent, consisted of 6 pairs of antigens, with spacings of ˜13 nm between antigens in a given pair, and greater than 20 nm between neighboring pairs. Based on the published spatial tolerance of antibodies¹⁹, we hypothesized this design would permit bivalent binding of a single antibody to each antigen pair, while preventing binding across neighboring pairs (FIGS. 3A-3B). The second variant, called DNB12-monovalent, consisted of all 12 antigen sites interspaced more than 20 nm apart, beyond the reported spatial tolerance of IgG antibodies for bivalent binding¹⁹. The original DNB12, which featured four triangular clusters of antigens spaced ˜16 to 18 nm apart, was renamed DNB12-intermediate, as it featured distances permissive to both bivalent and monovalent antibody binding. For IC assembly, we again incubated each design with increasing concentrations of the antigen-specific IgG antibody followed by analysis using GMSA (FIG. 3C). Consistent with our hypothesis, the DNB12-bivalent design assembled exclusively into monodisperse ICs across all antibody-antigen ratios tested, while the DNB12-monovalent design assembled exclusively into crosslinked ICs at antibody-antigen ratios of 1:1 and above. Interestingly, at sub-stoichiometric antibody-antigen ratios (i.e., 0.5), we observed a reverse in the extent of gel shift between the two designs. While neither design resulted in crosslinked ICs at this sub-stoichiometric ratio, DNB12-bivalent exhibited a greater upward gel shift compared to DNB12-monovalent, suggesting increased antibody binding. We speculate this is due to the antibodies' ability to bind bivalently on DNB12-bivalent, which is expected to have a lower dissociation constant compared to the monovalent binding of antibodies to antigens on DNB12-monovalent.

TEM characterization of the ICs assembled at the 1:1 antibody-antigen ratio further verified our findings (FIG. 3D). Specifically, we confirmed that DNB12-bivalent ICs were monodisperse, DNB12-monovalent ICs were exclusively crosslinked with sizes ranging from 0.2 to 1.5 μm, while DNB12-intermediate formed a mixture of both monodisperse and crosslinked ICs, as observed before. Together, these results collectively demonstrate a striking effect of antigen spacing on the physical structure of the IC. Namely, subtle alterations in antigen spacing of a few nanometers determine the outcome of antibody-antigen interactions into either monomeric, nanoscale ICs or insoluble aggregates.

### IC Binding and Uptake by FcγR-Expressing Immune Cells

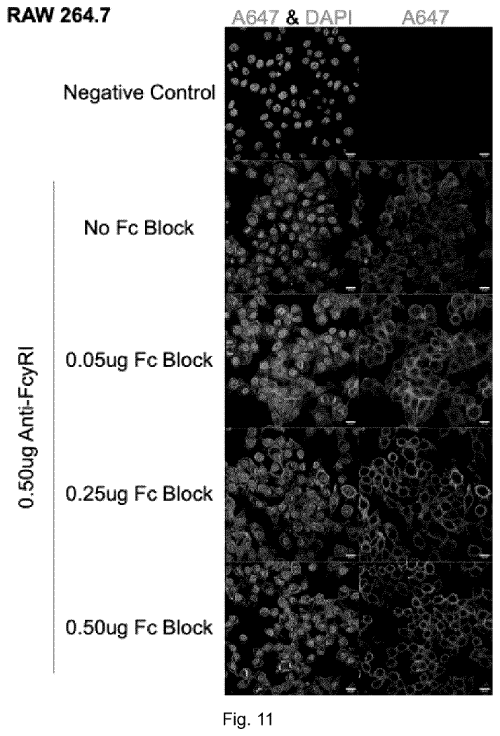

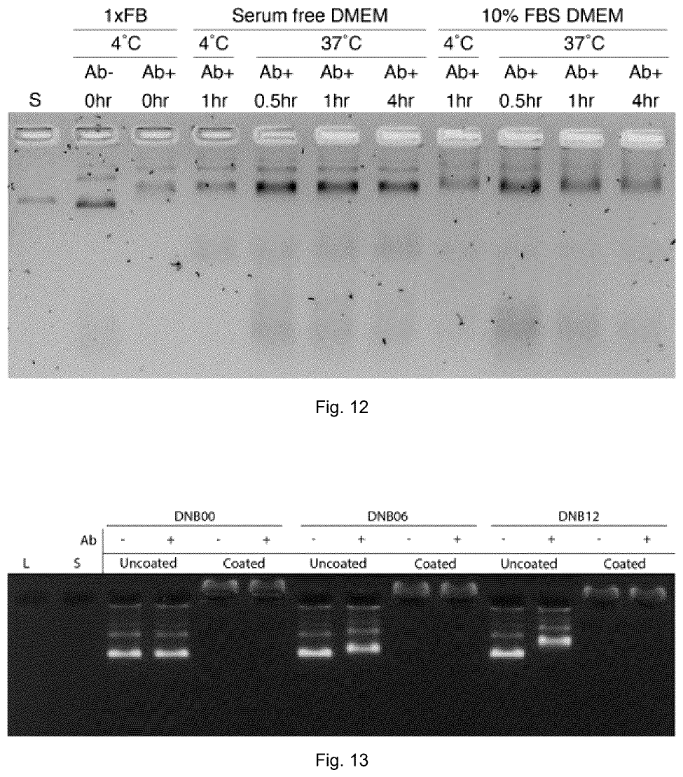

Antigens complexed by IgG antibodies are more efficiently bound and internalized by FcγR-expressing immune cells, such as macrophages and dendritic cells, while the rate of internalization has been shown to depend on IC physical parameters such as Fc density and spacing. This prompted us to ask how ICs assembled from DNA origami interact with FcγR-expressing cells as a function of their design. The ICs used in this study were assembled using the murine IgG2a isotype, which binds to FcγRI with high affinity, and to FcγRIV, FcγRIIIA, and FcγRIIB with lower affinity^{20,21}. Prior to cell experiments, we first verified that our model cell line, RAW 264.7 murine macrophages, expressed basal levels of FcγRI via immuno-labeling, consistent with earlier findings (FIG. 11)²². Additionally, we characterized the stability of the DNB-ICs following cold storage and incubation in cell culture media. Analysis by GMSA confirmed ICs maintained structural stability at 4° C. in storage buffer for at least 5 days (FIG. 9), and in both serum-free as well as 10% fetal bovine serum-supplemented culture media for at least 4 hours (FIG. 12). For detecting DNB-IC binding and uptake by cells, DNBs were labeled with six copies of Alexa Fluor®546 dye (AF546, FIGS. 4A-4B), and successful labeling was confirmed via co-localization of the dye and DNB by agarose gel electrophoresis (FIG. 13).

To investigate how antigen valency affects IC binding to FcγR-expressing cells, DNB00, DNB06, or DNB12-bivalent were assembled into ICs by incubation with antibodies at 1:1 antibody-antigen ratio. These three designs span 0, 6, or 12 copies of antigens and form monodisperse ICs at the 1:1 antibody-antigen ratio. RAW264.7 cells were incubated with 0.2 nM of each IC for 1 hour at 4° C. to block fluid phase uptake and isolate the effects of FcγR binding (FIG. 4A). IC binding was visualized by confocal fluorescence imaging. FIG. 4C shows representative single z-slices (0.64 μm thick) of confocal images taken at the approximate centre of the cell following the binding experiment. IC binding appeared as a uniform “ring” of fluorescence signals which outlined the cell boundaries, suggestive of cell-membrane binding. ICs assembled from DNB06 and DNB12 exhibited visually comparable signal intensities, while both appeared brighter than cells incubated with DNB00 and free IgG2a. Quantitative analysis of mean fluorescence intensities showed an increase in cell binding for both ICs assembled from DNB06 (1.3-fold) and DNB12-IC (1.5-fold), compared to their respective antibody-negative controls. Nevertheless, the difference in binding between DNB06-ICs and DNB12-ICs was small (˜1.1-fold), suggesting modest effects of antigen valency on IC binding to macrophages. This is consistent with the high affinity of FcγRI for monomeric IgG2a antibodies. Finally, cell binding of DNB00 was comparable to other antibody-negative controls (0.66- to 0.77-fold of ICs). These results suggest substantial non-FcγR-mediated interactions between the cell surface of macrophages and the surface of DNA origamis, independent of the decorated antigens and bound antibodies.

We aimed to strengthen Fc-FcγR interactions by masking macrophage interactions with the DNA origami surface. Previously, Ponnuswamy et al. established the use of oligolysine-poly(ethylene glycol) (K₁₀-PEG₅ₖ) to coat and protect DNA origamis from degradation²³, while PEG-based surface coatings are known to block nonspecific biomolecular and cell binding. Thus, we asked whether coating ICs with K₁₀-PEG₅ₖ could reduce DNA origami interactions with macrophage membrane molecules, while still allowing IC-bound antibodies to engage FcγRs. To test this, we coated DNB-ICs with K₁₀-PEG₅ₖ following published procedures and re-evaluated their binding to macrophages by confocal fluorescence imaging. Here, we observed a drastic reduction in the binding of DNB00 and antibody-negative controls to macrophages (FIG. 4E), while the respective ICs derived from the same designs maintained their ability to bind cells, as evidenced by the uniform ring of fluorescence signals around the cell membrane (FIG. 4E). Quantitatively the coated DNB00, DNB06, and DNB12 designs exhibited an average of 1.4- to 1.8-fold reduction in binding relative to their respective uncoated controls (FIGS. 4D, 4F), while the binding of coated and uncoated ICs remained unchanged. This translated into a 2.2- and 1.7-fold enhancement in the binding of coated DNB06-IC and DNB12-ICs compared to their respective antibody-negative controls. Altogether, our results demonstrate that antigen valency did not have a major effect on DNB-IC binding to macrophages, possibly since FcγRI is known to bind monomeric IgG2a with high affinity. These observations are consistent with prior results that IC valency affects the multimerization state of FcγR much more than binding. Additionally, DNA origami ICs interact with macrophages via both FcγR and non-FcγR interactions; the latter can be strengthened by coating DN-ICs with PEG-based surface coatings.

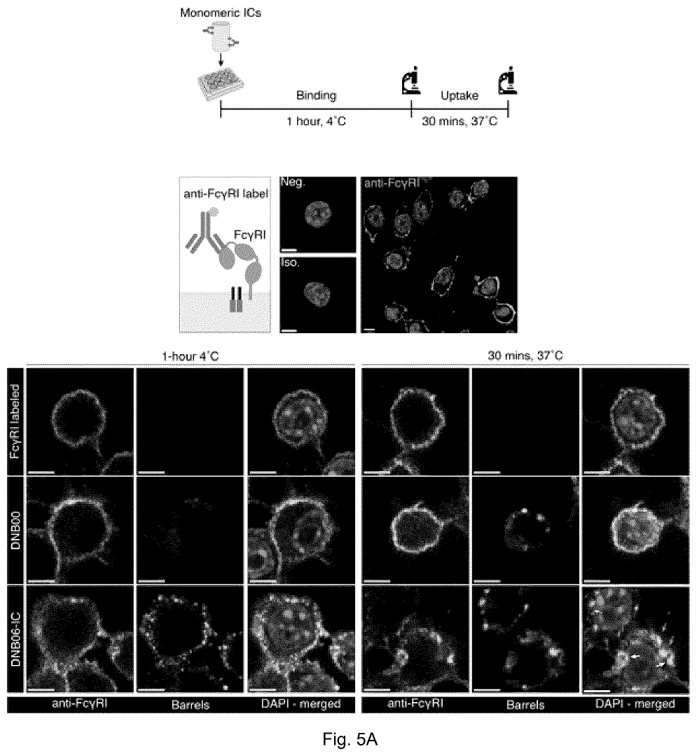

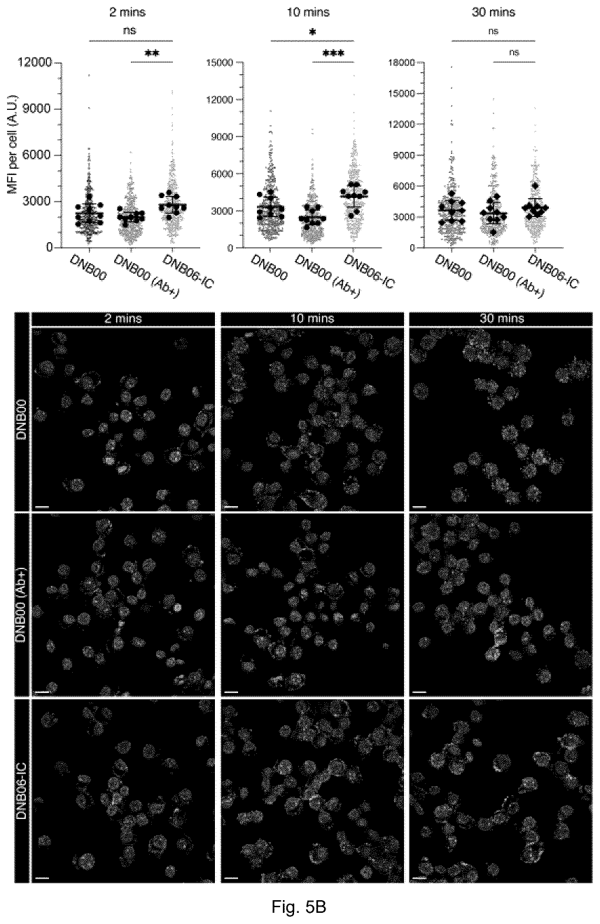

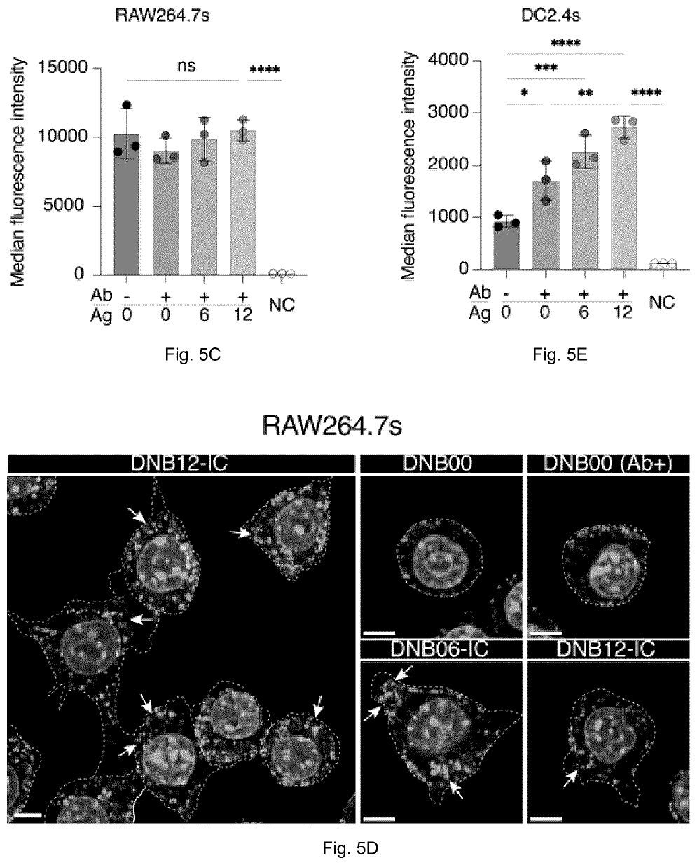

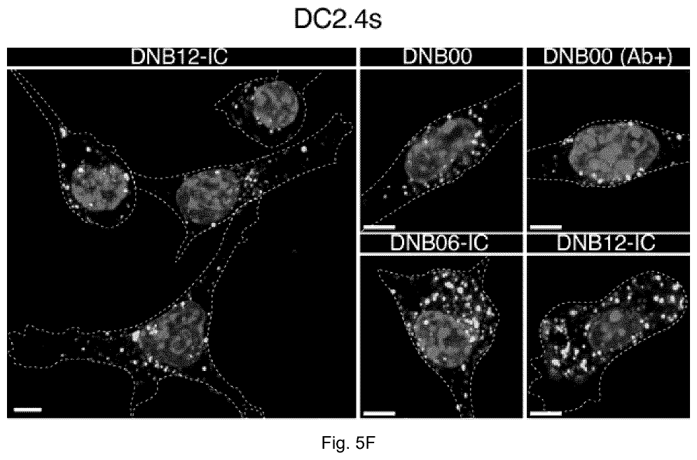

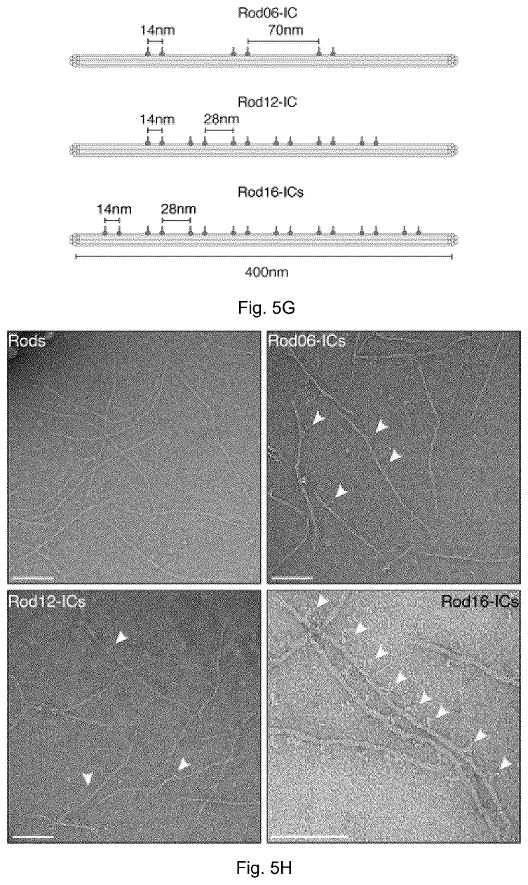

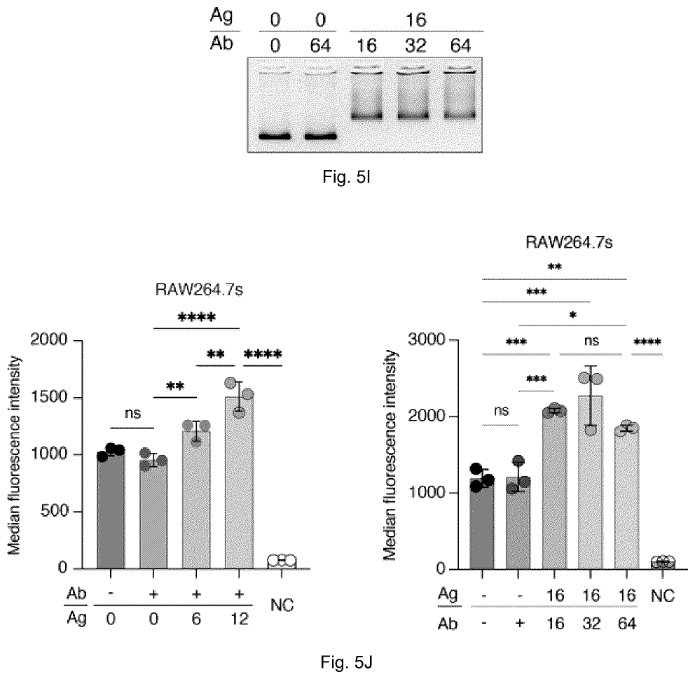

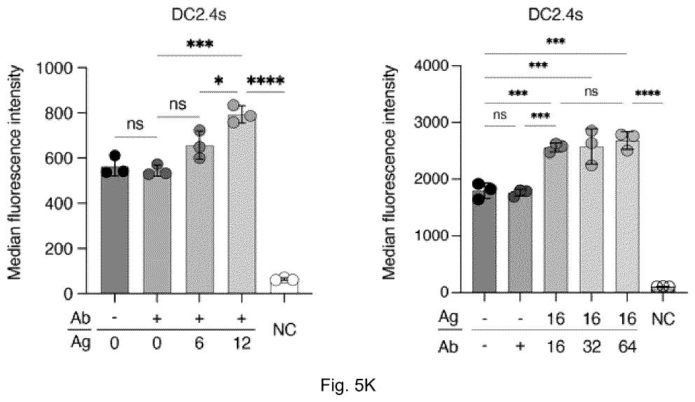

To further confirm FcγR-mediated IC-cell binding, we co-labeled FcγRIs using an AF647-tagged anti-FcγRI antibody following cell binding of AF546-tagged DNB-ICs (FIG. 5A). The expression of FcγRI appeared as a uniform ring on the cell membrane under confocal fluorescence imaging and co-localized with DNB-ICs, while DNB00 exhibited minimal binding. These samples were subsequently transferred to 37° C. for 30 minutes to induce uptake. Here we observed spatial rearrangement of the DNB06-ICs and FcγRI signals towards the cell interior (FIG. 5B). Moreover, we observed internalization (white arrows), reorganization (yellow arrows), and depletion (yellow arrows) of the FcγRI label indicative of FcγRI-mediated uptake, which were not observed in the DNB00 control.

Antibody-opsonization has been shown to accelerate uptake of foreign materials by macrophages. A prior study demonstrated that antibody-opsonized beads of similar size to our DNBs were trafficked to lysosomes within macrophages in 30 minutes²⁴. Thus, we asked whether DNB-ICs were internalized faster than their non-IC DNB controls (FIG. 5B). To test this, cells were incubated with 0.2 nM of either DNB06-ICs or DNB00 with free antibodies for 2, 10, and 30 minutes at 37° C. The amount of DNA origami within the cells was detected and quantified via origamiFISH²⁵. Distinct outer rings of fluorescence signals could be observed on cells as soon as 2 minutes following exposure to DNB06-ICs, but not on cells exposed to the DNB00 and free antibodies. At 10 minutes, cells exposed to DNB06-ICs exhibited greater number of puncta in the cell interior compared to cells exposed to DNB00 with free antibody. Interestingly, these differences in intracellular signal appeared to taper off by 30 minutes, and quantitative image analysis demonstrate no difference in fluorescence signals between cells exposed to DNB06-IC and DNB00 with free antibodies. These results suggest that FcγR-mediated interactions accelerate the internalization of DNB06-ICs by macrophages, while non-FcγR interactions, such as micropinocytosis or other receptor-mediated endocytosis, may account for the majority of uptake at later time points.

ICs with Size-, Shape- and Cell Type-Specific Uptake Patterns

Next, we looked to determine if IC antigen valency could impact the magnitude of uptake in macrophages. Accordingly, we incubated 1 nM of our AF-546 labelled monodisperse DNB-ICs with macrophages for 30 minutes to compare the total amount of uptake across designs (FIGS. 5C and 5D). Flow cytometry revealed that DNB06-ICs and DNB12-ICs had no significant differences in the magnitude of uptake compared to DNB00 with free antibody. This was surprising given the significant differences that were found in cell binding. This shows that monodisperse DNA nanoparticles whether opsonized or not, and regardless of their antigen valences are taken up to similar degrees by macrophages.

Since macrophages took up DNB-ICs and non-opsonized barrels to similar magnitudes we wondered whether dendritic cells were more selective. Dendritic cells (DCs) are also FcγR expressing phagocytes, however in various contexts they are known to be less efficient in particle uptake compared to macrophages²⁶. Thus, understanding whether they preferred opsonized DNs and if the valency further impacted their propensity to take up these ICs was of interest. Again, we incubated 1 nM of our AF-546 labelled monodisperse DNB-ICs with DC2.4 cells for 30 minutes to compare the total amount of uptake across designs (FIGS. 5E and 5F). Flow cytometry revealed that both DNB06-ICs and DNB12-ICs were taken up significantly more compared to DNB00 with free antibody (1.3 and 1.5-times more, respectively). Interestingly, this uptake assay also revealed that the magnitude of IC uptake was antigen valency dependent, as DNB12-ICs were taken up 1.2-times more compared to DNB06-ICs.

Studies have explored the notion that the shape of pathogens impact their immune cell internalization, and thus have investigated how shape and size of DNA nanostructures (DNs) influence their overall uptake into macrophages.

Endocytosis of membrane bound materials requires actin reorganization²⁷ and has been shown to be more efficient for spherical particles^{21,34}. In line with these findings, we have identified long filamentous DNA rods to have limited uptake in macrophages²⁵. Considering this, we considered whether opsonization would increase the internalization of a DNA nanostructure not readily taken up by macrophages. In doing so we looked to unveil shape specific factors that govern IC uptake in macrophages.

First, we synthesized rod-ICs using earlier established antigen spacings that resulted in monodisperse ICs. The DNA rods are 7 nm thick and 400 nm long, with 8 antigen pairs (16 antigens total) intra-spaced 14 nm apart to promote bivalent antibody binding (FIG. 5G).

After incorporating biotins onto the rods, samples were incubated with increasing molar ratios of antibody. IC formation was then evaluated using a GMSA (FIG. 5H). The gel reveals that non-biotinylated rods do not interact with IgG and that rod-16s undergo a band shift when bound by IgG to form rod-ICs. Additionally, rod-ICs undergo an observable shape change as seen via TEM compared to non-opsonized rods (FIG. 5I-5J).

Non-opsonized DNBs are taken up readily by macrophages as confirmed and previously established that DNs with low aspect ratios and greater compactness were preferentially suited for macrophage uptake²⁹. Despite this we do demonstrate that these complexes bind and are taken up through FcγRI engagement. To determine whether opsonization could increase the uptake of rods and provide further evidence that these engineered DN-ICs are taken up through FcγRI interactions we incubated the rod-ICs with increasing concentrations of IgG2a during a 30-minute uptake reaction. At a 1:1 antibody: antigen ratio the quantity of rod uptake peaked, with a 1.4-times increase in uptake (FIG. 5K-5L). Interestingly, as the concentration of antibody in excess increased, the magnitude of uptake decreased to that of the non-opsonized control indicating competition between free IgG and opsonized rod-ICs. This decrease in uptake further substantiates that DN-ICs are taken up specifically through Fc-FcγRI interactions.

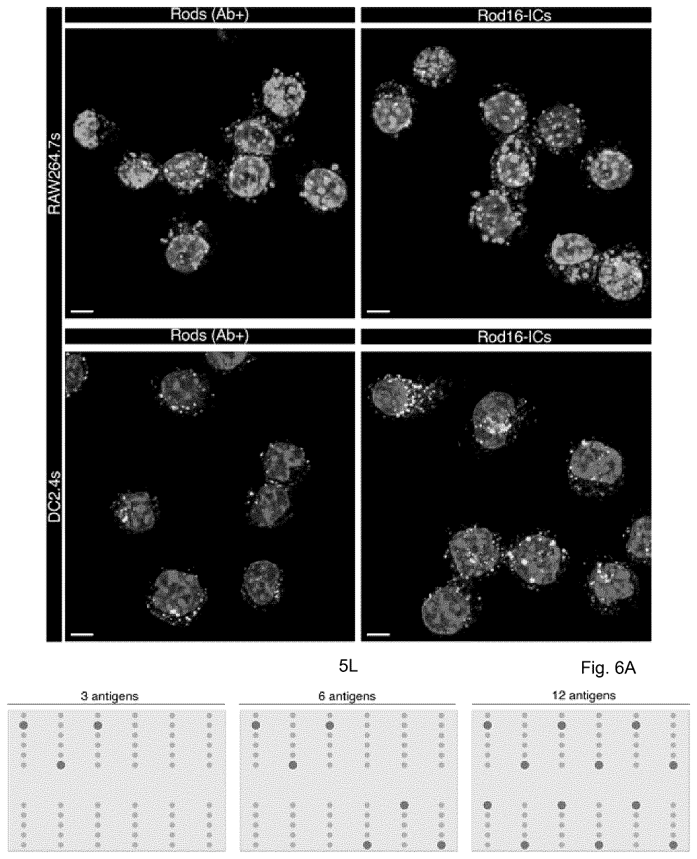

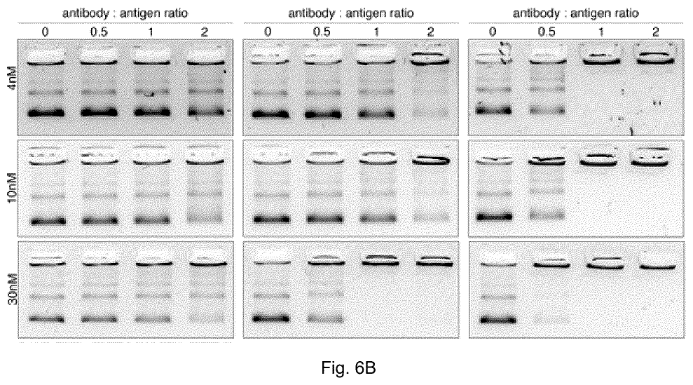

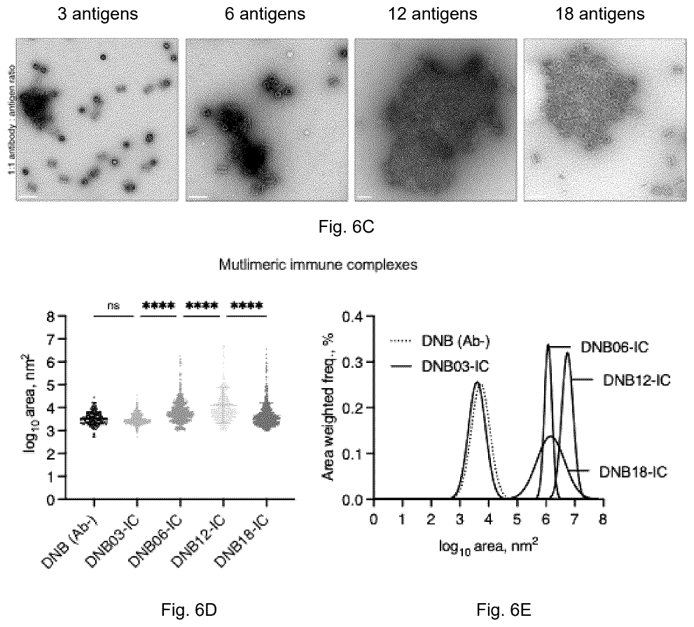

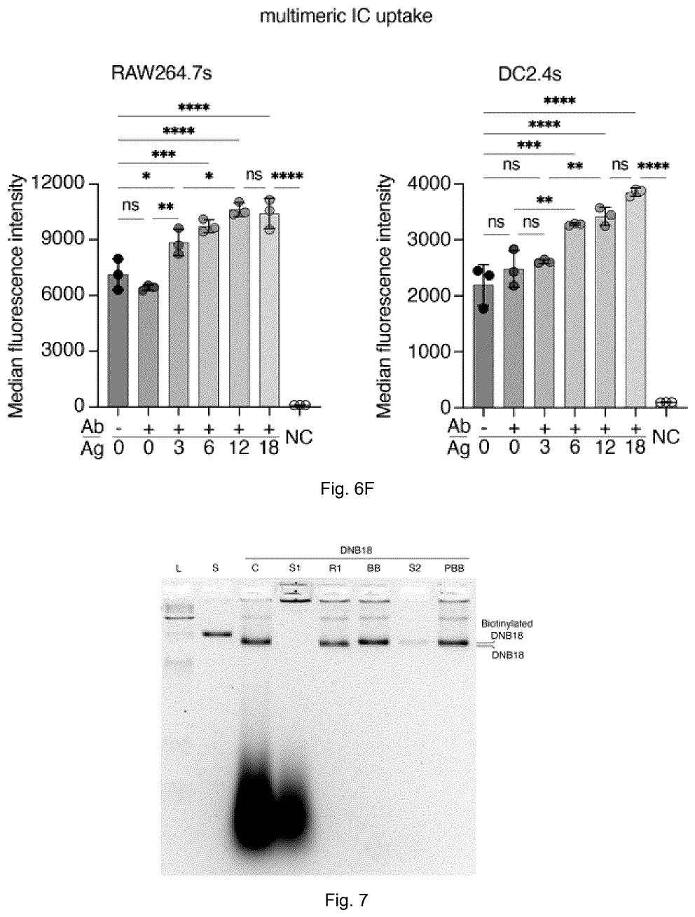

Next, we sought to use our design principles to create a library of IC designs that ranged in size. We hypothesized that tuning IC size would be possible by varying the valency of antigens while maintaining the intra-antigen spacing between designs (FIG. 6A). This was the case as increasing antigen valency led to an observed increase in DN cross-linking as characterized by the degree of aggregation in wells on GMSAs (FIG. 6B). To better understand the differences in size, DNB-ICs formulated at a 1:1 antibody-antigen ratio at 30 nM were observed under TEM (FIG. 6C). Next, images of the ICs were captured to measure observed differences in particle size as a function of antigen valency and spacing designs (FIG. 6D). Using prescribed antigen valency and spacings we show that the size of the immune complexes can be controlled, where the measured surface area of DNB03-ICm <DNB06-ICm <DNB18-IC<DNB12-ICm. The surface areas of the various IC designs ranged from 1800 nm² for monodisperse IC designs (DNB06-IC, DNB12-IC) to 3900 nm² for DNB03-ICm, to 1,193,988 nm² for DNB06-IC, to 5,584,702 nm² for DNB12-ICm and 1,452,111 nm² for DNB18-IC.

To understand how the size of immune complexes affects uptake into macrophages and DCs, we incubated each design separately at 1 nM for 30 minutes, with both RAW264.7 macrophages and DC2.4 dendritic cells. Previously, we showed that monodisperse DNB-ICs were not preferentially taken up by macrophages, however in this experiment we show that the larger the IC the greater the magnitude of uptake. Interestingly, this relationship is not fully maintained in dendritic cells, where we show that for DC2.4s, the difference in uptake seems most impacted by the antigen valency of the design as opposed to strictly size based as observed in macrophages. Overall, our results demonstrate that tailoring DN-IC size can be used to increase uptake into macrophages and dendritic cells.

### DISCUSSION

Synthetic immune complexes (ICs) were engineered by assembling IgG antibodies with antigens displayed on 3D nucleic acid origami scaffolds. The use of DNA origami allowed programmed spatial patterns of antigen with precise valency and spacing on custom nanoscale shapes. The spatial parameters of antigen display is a critical variable in determining the structure of the DNA origami-ICs (DN-ICs). Similarly, IC structure also depended on the ratio of antibodies to antigens. Increasing antibody ratios resulted in design-specific IC structures. Additionally, antigen spacings at maintained valences exploited IgG binding tolerances to provide design paradigms to control IC aggregation in solution. Inter-particle crosslinking was limited when intra-particle antigen pairs were spaced ˜13 nanometers (nm) apart, as ICs maintained monodispersity. Whereas, when antigens were spaced at distances larger than the binding tolerance of IgG, i.e. >20 nm apart, inter-particle crosslinking was induced. Taken together, the structural properties of DN-ICs can be controlled with organized antigen valences, spacings, and antibody or DN concentrations. To assess how the structural features of synthetic DN-ICs influence a ligand-receptor mediated biological function, we evaluated their respective uptake profiles in murine macrophages and dendritic cells. Here we showed that DN-ICs were taken up via FcgR interactions in a cell-type dependent manner compared to non-opsonized controls. Higher valency ICs also exhibited greater amounts of binding compared to lower valency designs. Using prescribed antigen valency and spacings we show that the size of the immune complexes can be controlled. The measured surface areas of the various IC designs ranged from 1800 nm² for monodisperse IC designs (DNB06-IC, DNB12-IC) to 3900 nm² for DNB03-ICm, to 1,193,988 nm² for DNB06-IC, to 5,584,702 nm² for DNB12-ICm and 1,452,111 nm² for DNB18-IC. The measured diameter of monodisperse DNB-ICs were 30 nm with a side-length height of 60 nm, whereas for our largest ICs, DNB12-ICm, which showed exclusively crosslinked ICs under TEM, the diameters of the ICs ranged from 0.2 to 1.5 μm. Thus, programming the antigen spacing allows us to engineer synthetic, soluble ICs that span 3 orders of magnitude in size. Our results demonstrate that the size of DN-ICs was influential in guiding increased uptake into macrophages and dendritic cells.

Overall, the present disclosure demonstrates control of IC structure using DNA nanotechnology and provides a platform to investigate FcγR immune responses to synthetic ICs. In a broader context, clinical immunotherapies such as intravenous immunoglobulin (IVIG) have had success in treating various diseases including auto-immune diseases by selectively engaging or blocking FcγR signaling³⁰; however, their efficacies are limited by valency and heterogeneity in donor IgG subtypes³¹. Alternatively, recombinant Fc-multimer engineering approaches have been developed, but they are similarly bottlenecked by laborious preparations and limited flexibility in engineering Fc valency and geometry³². Thus, the use of our nano-platform may unveil specific structural factors of ICs that control FcγR signaling and downstream activation in a variety of immune cells, which have applications in therapeutics and vaccines.

### Example 2—Immunogenic Profile of DN-ICs Exposed to Macrophages

First, we characterized the cell morphology of RAW264.7s cells exposed to 1 nM of the DNA barrel ICs and respective controls for 8, 16, and 24 hrs.

### Experimental Details:

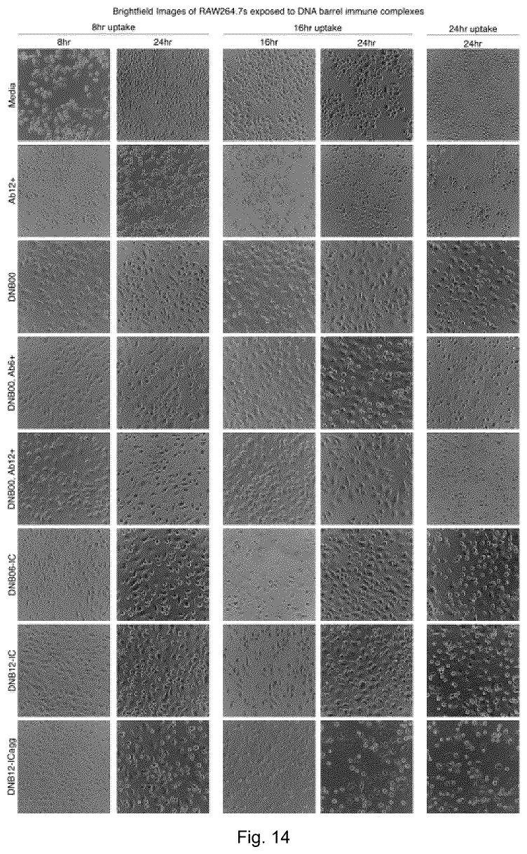

100k RAW264.7s cells were seeded in individual wells on a 24 well plate and pre-treated with IFN-gamma for 16 hours. Media was replaced with fresh media and left to equilibrate for 2 hours prior to cell uptake. 1 nM of material was introduced to RAW264.7s cells for 8, 16, and 24 hours. At 8- and 16-hours uptake media was removed and replaced with fresh media. For all samples brightfield microscopy images were acquired to assess morphological changes at the designated exposure times and following media replacement up to the 24-hour mark. See FIG. 14.

### Conclusions:

IFN-gamma induces macrophages to become activated with a sprawled-out morphology.
When exposed to Ab12+ of IgG2a, the antibody only control, which has the same antibody concentration as the IC samples formulated with 12 antigens, the cells revert to a non-activated morphology with almost a complete spherical morphological population.
All samples present with DNA origami barrels either without antibody, with free antibody, or formulated as ICs cause an increase in total cell surface area. The cells present with a ‘dendritic-like’ morphology. Cells become even more sprawled out than the media control and form long outreaching dendrites that make connections with the culture surface and other cells.
This morphology can be observed as early as 8 hours and persists in the IC positive samples for up to 24 hrs during or following an 8- and 16-hour exposure with limited cell death. This ‘dendritic-like’ morphology is less prominent in the barrel only sample and the barrel samples co-incubated with free IgG. Whereas at the 24-hours mark, large sprawled out macrophages are more persistent in the DNB-IC samples. At the 24-hours mark cell death was clearly observed in the Ab12+ sample, it was also observed in the DNB samples co-incubated with free IgG but to a lesser extent.

To better understand these morphological differences. Enzyme linked immunosorbent assays (ELISAs) were performed on the cell supernatants. ELISAs for three separate cytokines were measured. The three cytokines measured, IFN-beta, TNF-alpha, and IL-6 play key roles in regulating immune response. Both TNF-alpha and IL-6 are involved with activating T cells and B cells. It is known that these pro-inflammatory cytokines drive an inflammatory response during infection, tissue injury, autoimmune diseases, and cancer. IFN-beta has been found to enhance antigen presentation and promote the differentiation of T cells into effector cells.

### Experimental Details:

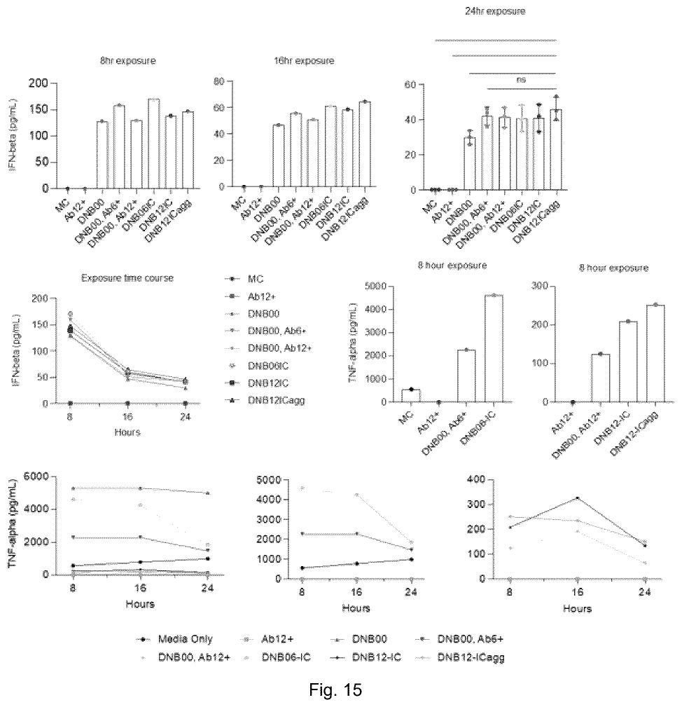

100k RAW264.7s cells were seeded in individual wells on a 24 well plate and pre-treated with IFN-gamma for 16 hours. Media was replaced with fresh media and left to equilibrate for 2 hours prior to cell uptake. 1 nM of material was introduced to RAW264.7s cells for 8, 16, and 24 hours. At 8- and 16-hours uptake supernatant was removed and replaced with fresh media. For all samples and timepoints supernatants were acquired to perform ELISAs. See FIG. 15.

### Conclusions:

In one biological replicate, for the 8-hours and 16-hours exposure to ICs and their respective controls, IFN-beta production is similar across all samples.
Across three biological replicates for the 24-hours samples exposed to ICs and their respective controls, IFN-beta production induced by DNB12-ICagg was significantly different than DNBO0, but not compared to DNB00 with free antibody.
For all samples IFN-beta production peaked at 8 hours
In one biological replicate, following 8-hours exposure to DNB06-IC, TNF-alpha production was double that of its respective control, DNA barrels co-incubated with an equimolar amount of free IgG (DNB00, Ab6+).
Similarly, this was the case for DNB12-IC and DNB12-ICagg when compared to its respective control DNB00, Ab12+. DNB12-ICagg produced more TNF-alpha compared to the monodisperse DNB12-IC.
TNF-alpha was reduced by ˜10-fold in the 12 antigen designs compared to DNB06-IC.
In one biological replicate, following 8-hours exposure to DNB-ICs and respective controls, IL-6 production was highest for the sample incubated with only barrel (DNBO0). This was 2.7× more than DNB06-IC, and 7× more than DNB12-IC and DNB12-ICagg. The aggregated DNB06-ICagg produced 1.5× more IL-6 than the monodisperse IL-6.

### Additional Materials Characterization:

To better determine the effect that Ag valency and spacing contributes to the Ab composition and valency on the DNBs we developed an assay to quantify the number of IgG antibodies per structure. To do so DNBs were first labelled with AF546. Following labelling, DNB-ICs were formulated with AF647 primary labeled anti-biotin IgG (labeled in house). The 1:1 ratio was used. Samples were then purified with PEG precipitation to remove any unbound IgG. The total number of antibodies per IC were then quantified using a fluorescent plate reader and measured against a known standard curve for both the Ab and DNB concentration. ICs were formulated at 30 nM.

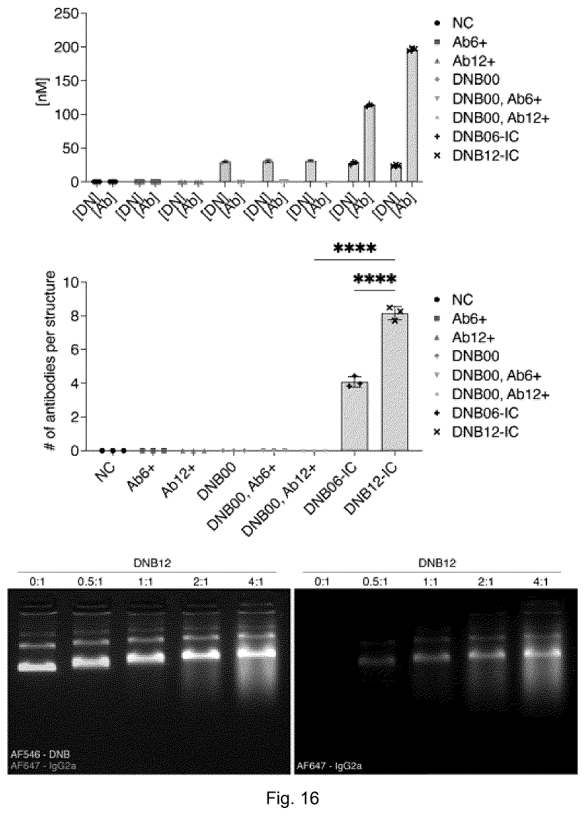

See FIG. 16.

### Conclusions:

There was a total of 4 antibodies per structure for DNB06-IC and a total of 8 antibodies per structure for DNB12-IC.
This result show that at the 30 nM formulation concentration, at a 1:1 antigen to antibody ratio, 50% of the Ag sites are occupied by antibodies bound through bivalent interactions and the other 50% of the Ag sites are occupied by antibodies bound through monovalent interactions.
As a result of the prescribed antigen spacings and valences on the surface of these barrel designs, this two-state, full occupancy, antibody binding equivalency prohibits aggregation.
This is further confirmed by the gel as increasing labeled-Ab concentrations still do not cause aggregation, indicative that these cross-linking events are prohibited by the designated Ag valences and spacings in DNB06 and DNB12.

### Secretion of RAW 264.7 Macrophages in Response to DNA Scaffolded Immune Complexes

Respective samples were exposed to RAW 264.7 macrophages for 8 hours (1 nM of DNA barrels). Following exposure supernatants were collected and IL-6 ELISA was performed. Negative control is media only. IgG2a, cells were exposed to 12 nM of free IgG2a (equimolar amount to [1:1] antibody-antigen samples). DNB00, +IgG2a indicates DNA barrels without antigen co-incubated with free IgG2a. DNB12-IC, IgG2a indicates monomeric DNA barrel immune complexes with 12 antigens. Brackets indicate [antibody:antigen] ratio, 1 nM of monomeric DNB12-IC at [1:1] has 12 nM of IgG2a.

### Result:

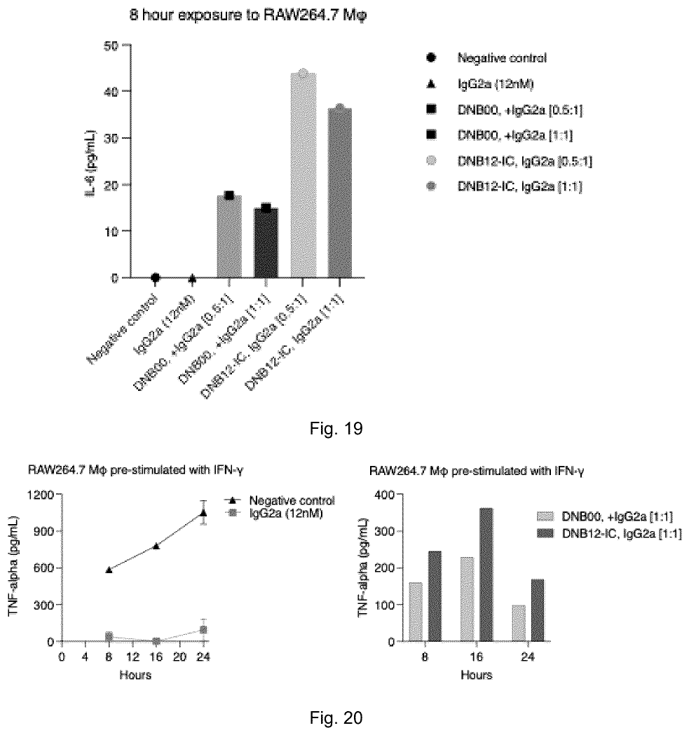

With reference to FIG. 19, macrophages secrete higher amounts of IL-6 when exposed to opsonized DNA origami compared to DNA origami co-incubated with equimolar amounts of free IgG2a. When DNA origami is presented as an immune complex, macrophages respond with a higher IL-6 secretion compared to a non-opsonized target.

Macrophages modulate their pro-inflammatory response based on the opsonization state of DNA origami. As well this inflammatory response is impacted by the concentration of antibodies to the concentration of binding epitopes on the target.

### TNF-α Secretion of IFN-γ Stimulated RAW 264.7 Macrophages in Response to DNA Scaffolded Immune Complexes

Respective samples were exposed to IFN-γ stimulated RAW 264.7 macrophages for 8, 16, and 24 hours (1 nM of DNA barrels). Following exposure supernatants were collected and TNF-α ELISA was performed. Negative control is media only. IgG2a, cells were exposed to 12 nM of free IgG2a (equimolar amount to [1:1] antibody-antigen samples). DNB00, +IgG2a indicates DNA barrels without antigen co-incubated with free IgG2a. DNB12-IC, IgG2a indicates monomeric DNA barrel immune complexes with 12 antigens. Brackets indicate [antibody:antigen] ratio, 1 nM of monomeric DNB12-IC at [1:1] has 12 nM of IgG2a.

### Result:

As shown in FIG. 20, macrophages secrete higher amounts of TNF-α when exposed to opsonized DNA origami compared to DNA origami co-incubated with equimolar amounts of free IgG2a.

By 24 hours TNF-α secretion declines but they remain higher for the DNA nanostructures formulated as immune complexes compared to the same DNA nanostructures co-incubated with free IgG2a.

Macrophages modulate their pro-inflammatory response based on the opsonization state of DNA origami. When DNA origami is presented as an immune complex, macrophages respond with a higher TNF-α secretion compared to a non-opsonized target.

FIGS. 19 and 20 demonstrate that the nucleic acid immune complexes of the present disclosure stimulate macrophage cytokine responses, and that the response requires the formation of such immune complexes.

### Incorporation of a Cargo Payload in the IC

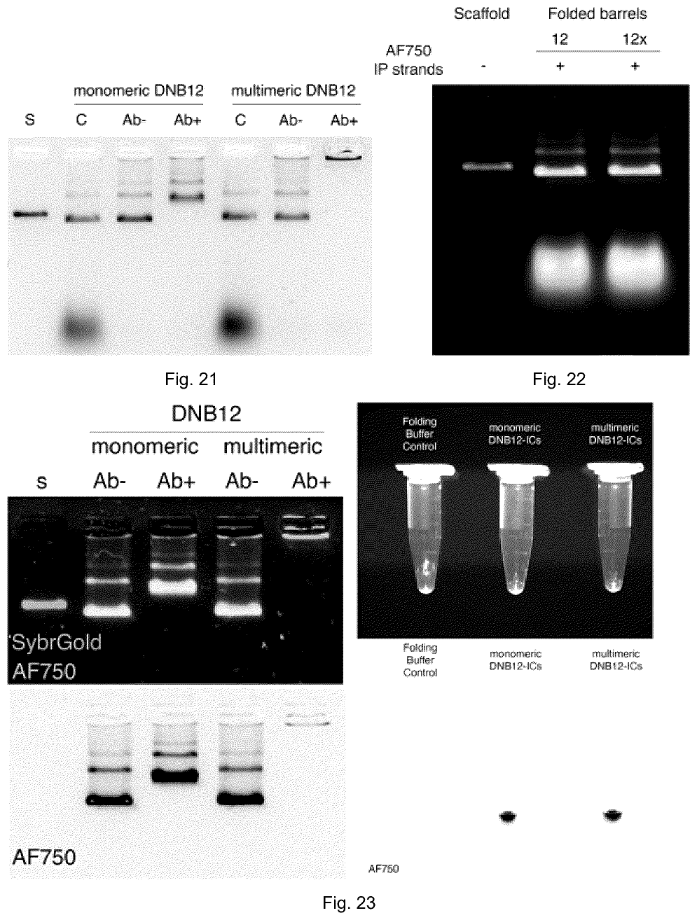

Presented in FIGS. 21 to 23 are engineered synthetic immune complexes with tracking dyes incorporated directly into the inner lumen of the DNA barrel scaffold. This demonstrates the ability to synthesize defined ICs with a cargo/payload in the same unit.

FIGS. 21-22 demonstrate a variant of the nucleic acid immune complexes of the present disclosure where the antigens are patterned via an alternate chemistry, i.e. direct incorporation of into the staple strands of the structure.

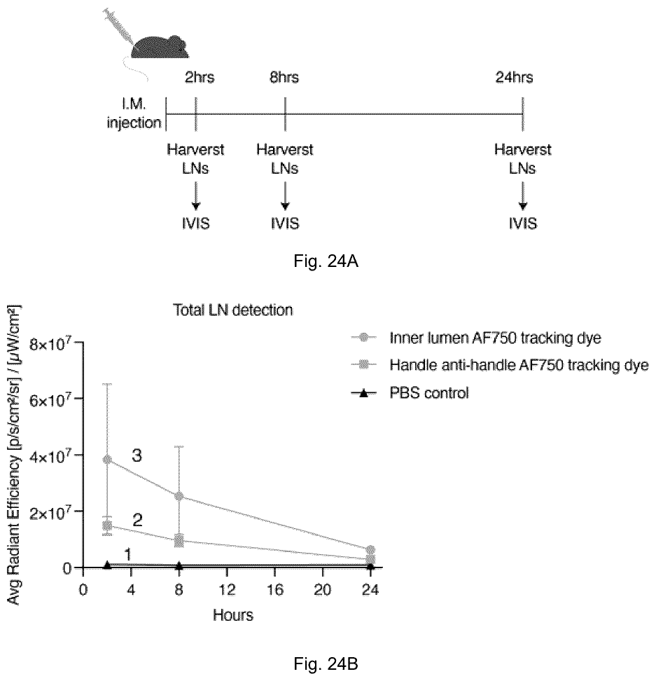

FIG. 23 demonstrates incorporation of payloads (i.e. a near-infrared dye) into the nucleic acid immune complexes. This shows that nucleic acid immune complexes can be used to carry small molecule cargoes, such as contrast agents for diagnostic imaging.

### Conjugation of Tracking Fluorophore to the Inner Lumen Improves Signal Detection in Tissues Over Time

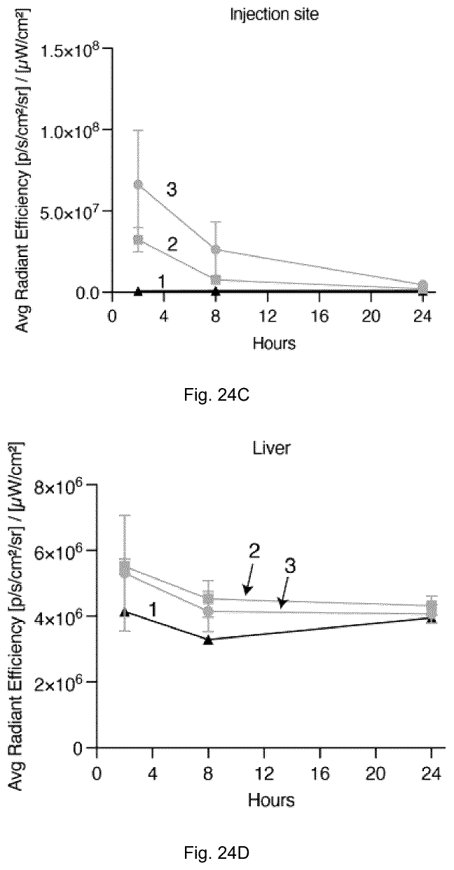

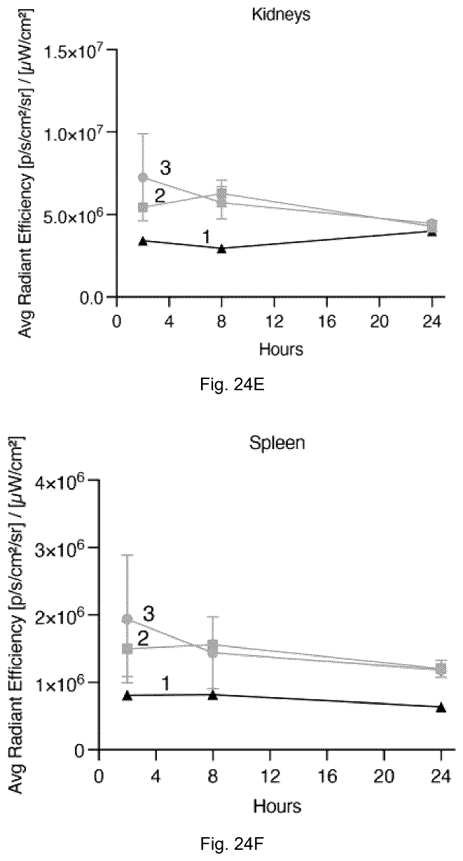

To assess how the position and conjugation strategy of the tracking fluorophore impacts our ability to detect the biodistribution of DNA nanostructures in vivo, mice were intramuscularly injected with DNBs bearing AF750 dye either within the inner lumen or on outer surface anti-handles, and tissues were analyzed over time (FIG. 24A). Internal conjugation of the fluorophore improved signal detection in draining lymph nodes compared to externally labeled counterparts, with sustained signal observed up to 24 hours (FIG. 24B). Similarly, conjugation of the fluorophore to the inner lumen improved signal detection at the injection site across all time points. Both designs shared comparable off-target profiles in liver (FIG. 24D), kidneys (FIG. 24E), and spleen (FIG. 24F). These results indicate that our new DNB iteration is better suited for in vivo applications as the conjugation strategy enhances signal detection without affecting systemic biodistribution or clearance.

DNA Barrel Scaffolded ICs Co-Functionalized with OVA Antigen

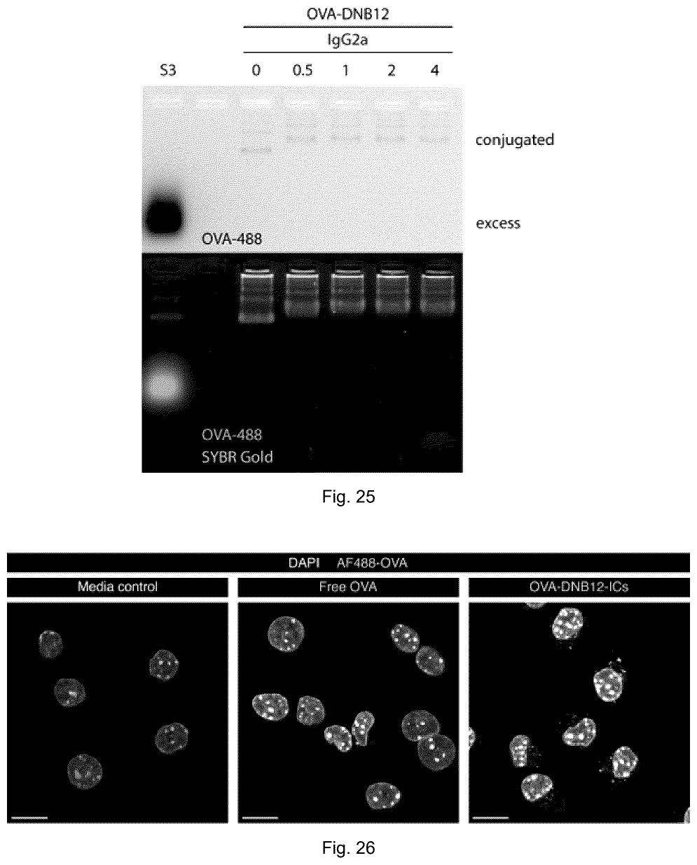

OVA-DNB12 was incubated with increasing concentrations of anti-biotin IgG2a antibody, titrated relative to the number of biotin moieties on the DNA barrel. The indicated molar ratios (0, 0.5, 1, 2, 4) represent IgG2a to antigen ratio. Immune complex (OVA-DNB12-IC) formation was assessed by agarose gel electrophoresis. As illustrated in FIG. 25, the band shift indicates successful binding of IgG2a to the biotinylated DNA scaffold bearing OVA.

FIG. 24 demonstrate use of the near-infrared contrast agent functionalized nucleic acid immune complexes of the present disclosure for in vivo animal imaging, specifically tracking the biodistribution of the material into major organs.

DNA Barrel Scaffolded ICs Co-Functionalized with OVA Antigen

FIG. 25 demonstrate incorporation of larger protein payloads (i.e. ovalbumin) into the nucleic acid immune complexes of the present disclosure. This shows that nucleic acid immune complexes can be used to carry larger macromolecules such as whole protein antigens and therapeutics.

Uptake of DNA Barrel Scaffolded ICs Co-Functionalized with OVA Antigen in RAW264.7s

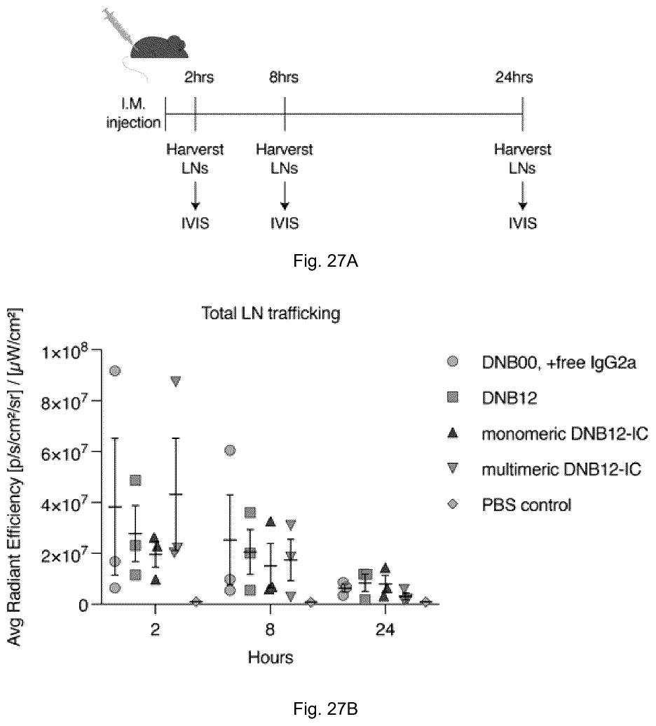

RAW 264.7s macrophages were incubated with OVA-DNB12-ICs or an equimolar amount of free OVA for 1 hour. Uptake was assessed via confocal microscopy. FIG. 26 illustrates the uptake of DNA barrel scaffolded ICs co-functionalized with OVA antigen in RAW264.7s.

FIG. 26 illustrates the ability of the nucleic acid immune complexes of the present disclosure to enhance cellular uptake of the ovalbumin cargo via receptor-mediated uptake

Monomeric Immune Complexes Reduce Variability in Lymph Node Trafficking while Maintaining Consistent and Comparable Biodistribution Over Time

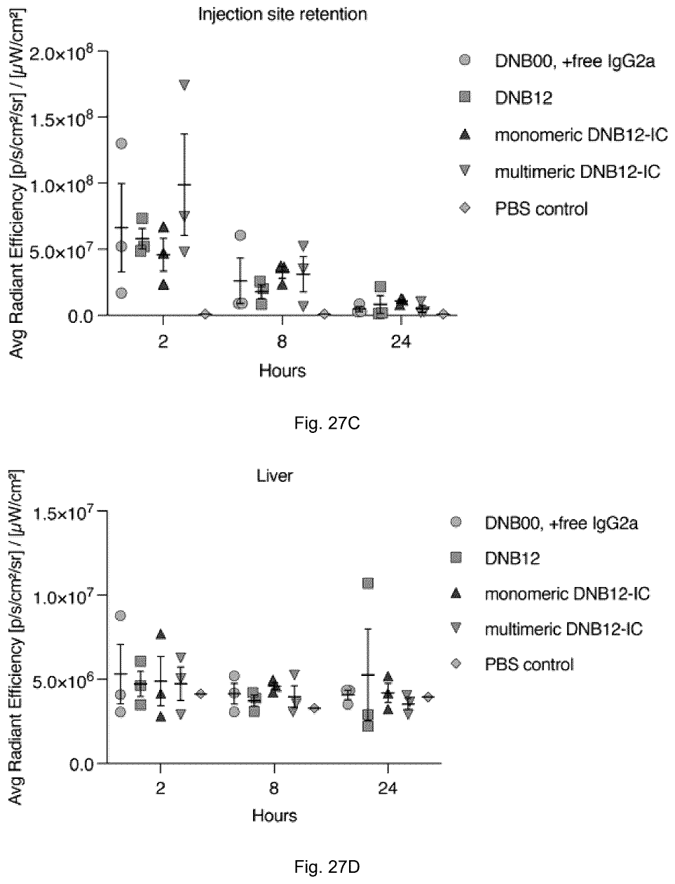

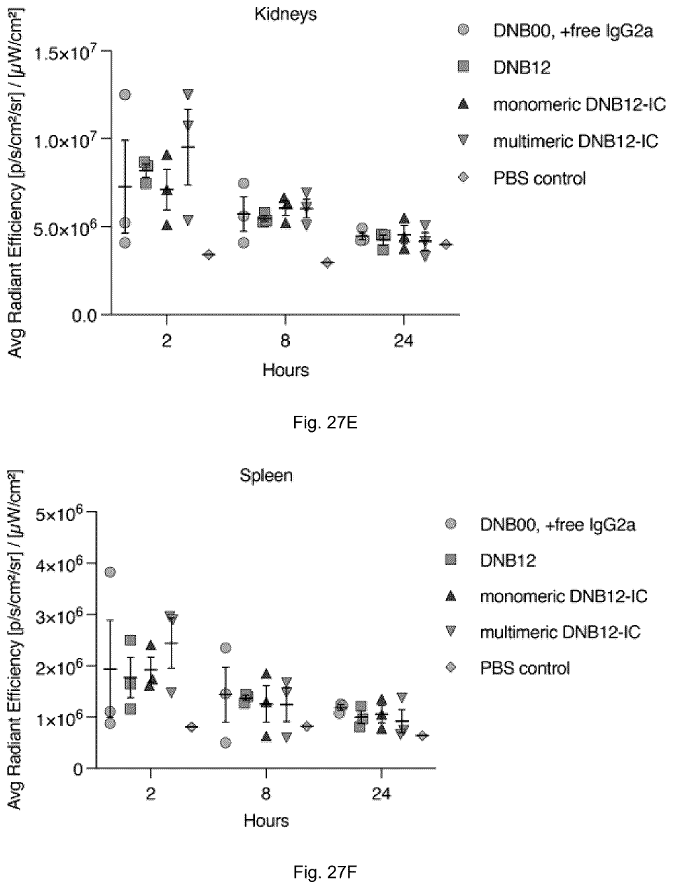

To evaluate the biodistribution of DNA scaffolded immune complexes (DNB12-ICs), mice were administered intramuscular injections of various DNB formulations, and tissues were analyzed at 2-, 8-, and 24-hours post-injection by IVIS imaging (FIG. 27A). Interestingly, multimeric DNB12-ICs exhibited enhanced accumulation in draining lymph nodes compared to monomeric DNB12-ICs, DNB12, or DNB00 with free IgG2a (FIG. 27B), at the 2-hour time point. However, this increase in accumulation as measured by IVIS was not significant and both DNB00 co-administered with equimolar amounts of IgG and multimeric DNB12-ICs had noticeably larger variance to that of monomeric DNB12-ICs. Monomeric DNB12-ICs reduced variability in lymph node trafficking and yet maintained similar levels of lymph node accumulation across all time points, including greater accumulation at 24-hours compared to its multimeric counterpart. Injection site retention (FIG. 27C) and off-target accumulation in liver (FIG. 27D), kidneys (FIG. 27E), and spleen (FIG. 27F) were comparable across all groups, with signal decreasing over time in all tissues. This data suggests that engineering immune complexes as monodisperse, homogeneous formulations improve consistency in lymph node delivery while maintaining a predictable biodistribution profile.

### Biodistribution of DNA Scaffolded Immune Complexes

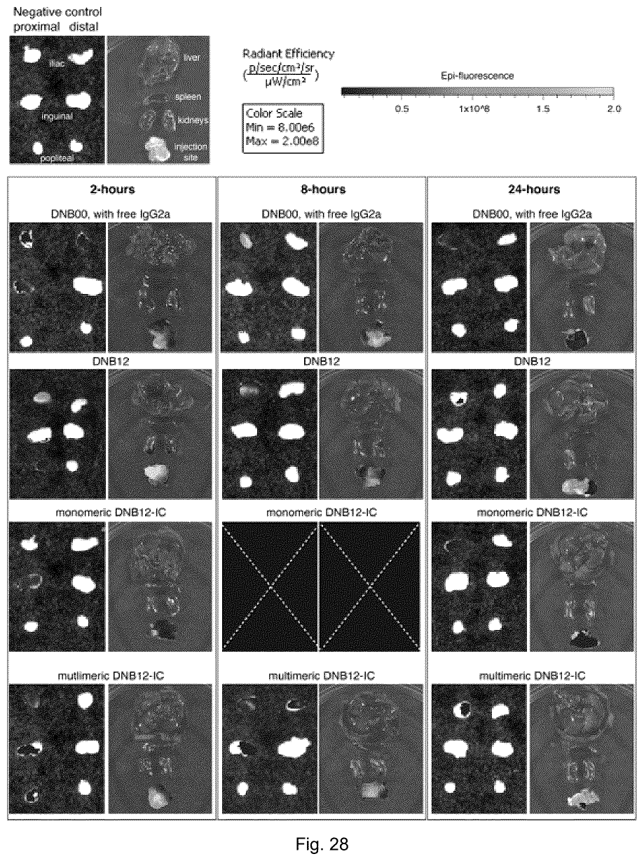

7-10-week-old female C57BU6 mice were intramuscularly injected in the right caudal thigh with 100 nM of the respective DNA nanostructure formulations. At 2-, 8-, and 24-hours mice were sacrificed, and lymph nodes and tissues were harvested to be imaged on IVIS. Results are shown in FIG. 28.

### Immune Complex Dispersity Controls Lymph Node Penetration and Distribution of

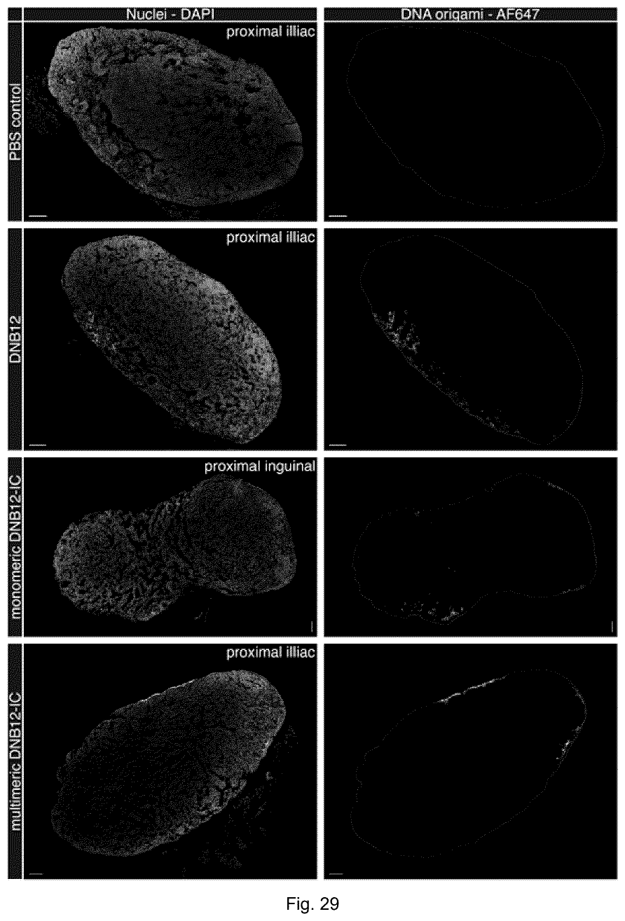

Cargo 7-10-week-old female C57BU6 mice were intramuscularly injected in the right caudal thigh with 100 nM of the respective DNA nanostructure formulations. At 2-hours mice were sacrificed, and lymph were sectioned to be stained via origamiFISH for confocal analysis. Results are shown in FIG. 29.

### Immune Complex Dispersity Controls Lymph Node Penetration and Distribution of Cargo

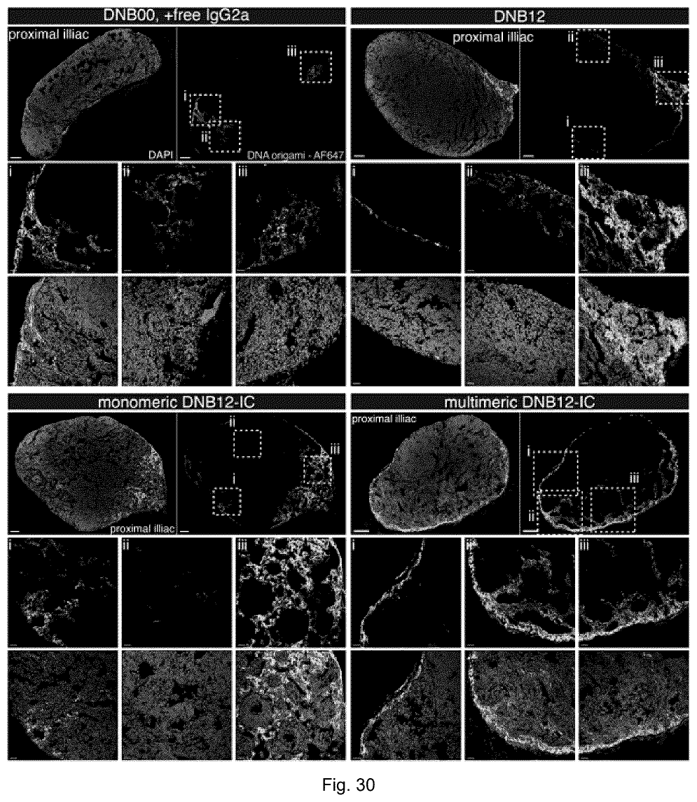

7-10-week-old female C57BU6 mice were intramuscularly injected in the right caudal thigh with 100 nM of the respective DNA nanostructure formulations. At 2-hours mice were sacrificed, and lymph were sectioned to be stained via origamiFISH for confocal analysis. Results are shown in FIG. 30.

Immune complex dispersity determines lymph node distribution. As expected, the PBS control shows no signal detection of DNA nanostructures in the lymph node. In FIG. 30 it can be observed that barrels co-administered with free IgG2a trafficked to the lymph nodes. Signal distribution seems to form at one major focal point on the lymph node and looks to diffuse inward from an intense signal on the lymph node periphery (FIG. 30. DNB00, i & ii). The origamiFISH signal looks to be diffusing inward from an afferent lymphatic vessel on the periphery of the lymph node indicative of passive lymph drainage and trafficking. Whereas, at the opposite side of the node (DNBO0, iii) particles are present on the interior of the node without signal on the capsule lining, indicative of trafficking to the subcapsular regions. Similarly, in both FIGS. 29 & 30, signal from DNB12 seems to traffic around the capsule (FIG. 30. DNB00, i) and accumulate in a region of the lymph node (FIG. 30. DNB00, iii) where it can be observed that particle signal diffuses along the subcapsular sinus regions (FIG. 30. DNB00, ii).

In both FIGS. 29 and 30 noticeable penetration within the lymph node (away from the LN perimeter) can be observed (FIG. 30. monomeric, i, ii, iii) in the monomeric IC samples. This signal is distinct as it forms away from any intense signal that accumulates along the capsules and can be observed as large clusters of puncta deep within both the proximal inguinal (FIG. 29) and the proximal iliac (FIG. 30. monomeric, ii) at the 2-hour time mark. Moreover, intense particle signal can be observed surrounding DAPI stained clusters that resemble follicles with some particles reaching the centers (FIG. 30. monomeric, iii). At 2 hrs monomeric ICs readily penetrate the lymph node compared to the previous designs and their multimeric IC counterpart. In both FIGS. 29 and 30 multimeric-ICs reach the lymph node in 2 hours however, a large proportion of their signal remains trapped along the capsule region. In FIG. 29, intense signal is present along the lymph node peripheries with no to minimal signal penetrating the tissue. In FIG. 30, there is a much greater concentration of particles, however, again the signal is largely distributed across the lymph node capsule. In fact, it can be observed passively leaking through afferent lymphatic vessels with proportions of the signal concentrating in the subcapsular sinus region (FIG. 30. multimeric, ii & iii) and only limited clusters penetrating similarly to monomeric-ICs (FIG. 30. multimeric, i). Overall, formulating synthetic immune complexes as monomeric, homogeneous improves lymph node distribution, increasing delivery efficiency and improving targeting.

FIGS. 27-30 show in vivo targeting of tissues using nucleic acid immune complexes of the present disclosure. These figures show that the different artificial IC designs of the present disclosure can achieve unique patterns of distribution and accumulation within the lymph nodes. This ability to target different areas of the lymph nodes using immune complexes has not been seen/shown before, but it holds significant implications for vaccine and immunotherapy design. This is a novel capability of the present technology that we are now able to demonstrate.

### REFERENCES

1. loan-Facsinay, A. et al. FcγRI (CD64) Contributes Substantially to Severity of Arthritis, Hypersensitivity Responses, and Protection from Bacterial Infection. Immunity 16, 391-402 (2002).
2. Lux, A., Yu, X., Scanlan, C. N. & Nimmerjahn, F. Impact of Immune Complex Size and Glycosylation on IgG Binding to Human FcγRs. J. Immunol. 190, 4315-4323 (2013).
3. Ortiz, D. F. et al. Elucidating the interplay between IgG-Fc valency and FcγR activation for the design of immune complex inhibitors. Sci. Transl. Med. 8, (2016).
4. Robinett, R. A. et al. Dissecting FcγR Regulation Through a Multivalent Binding Model. Cell Syst. 7, 41-48.e5 (2018).
5. Chen, H. et al. Detection and functional resolution of soluble immune complexes by an FcγR reporter cell panel. EMBO Mol. Med. 14, e14182 (2022).
6. Tan, Z. C. et al. Mixed IgG Fc immune complexes exhibit blended binding profiles and refine FcR affinity estimates. Cell Rep. 42, 112734 (2023).
7. Kim, M.-Y. et al. Novel vaccination approach for dengue infection based on recombinant immune complex universal platform. Vaccine 33, 1830-1838 (2015).
8. Phoolcharoen, W. et al. Expression of an immunogenic Ebola immune complex in Nicotiana benthamiana. Plant Biotechnol. J. 9, 807-816 (2011).
9. Rothemund, P. W. K. Folding DNA to create nanoscale shapes and patterns. Nature 440, 297-302 (2006).
10. Dietz, H., Douglas, S. M. & Shih, W. M. Folding DNA into Twisted and Curved Nanoscale Shapes. Science 325, 725-730 (2009).
11. Hahn, J., Chou, L. Y. T., Sorensen, R. S., Guerra, R. M. & Shih, W. M. Extrusion of RNA from a DNA-Origami-Based Nanofactory. ACS Nano 14, 1550-1559 (2020).
12. Douglas, S. M. et al. Self-assembly of DNA into nanoscale three-dimensional shapes. Nature 459, 414-418 (2009).
13. Douglas, S. M., Bachelet, I. & Church, G. M. A logic-gated nanorobot for targeted transport of molecular payloads. Science 335, 831-834 (2012).
14. Monferrer, A. et al. DNA origami traps for large viruses. Cell Rep. Phys. Sci. 4, 101237 (2023).
15. Tseng, C. Y., Wang, W. X., Douglas, T. R. & Chou, L. Y. T. Engineering DNA Nanostructures to Manipulate Immune Receptor Signaling and Immune Cell Fates. Adv. Healthc. Mater. 11, e2101844 (2022).
16. Veneziano, R. et al. Role of nanoscale antigen organization on B-cell activation probed using DNA origami. Nat. Nanotechnol. 15, 716-723 (2020).
17. Wickham, S. F. J. et al. Complex multicomponent patterns rendered on a 3D DNA-barrel pegboard. Nat. Commun. 11, 5768 (2020).
18. Lee, K. Z. et al. Engineering Tobacco Mosaic Virus and Its Virus-Like-Particles for Synthesis of Biotemplated Nanomaterials. Biotechnol. J. 16, 2000311 (2021).
19. Shaw, A. et al. Binding to Nanopatterned Antigens is Dominated by the Spatial Tolerance of Antibodies. Nat. Nanotechnol. 14, 184-190 (2019).
20. Unkeless, J. C. & Eisen, H. N. Binding of monomeric immunoglobulins to Fc receptors of mouse macrophages. J. Exp. Med. 142, 1520-1533 (1975).
21. Sears, D. W., Osman, N., Tate, B., McKenzie, I. F. & Hogarth, P. M. Molecular cloning and expression of the mouse high affinity Fc receptor for IgG. J. Immunol. 144, 371-378 (1990).
22. Verma, A. et al. Analysis of the Fc Gamma Receptor-Dependent Component of Neutralization Measured by Anthrax Toxin Neutralization Assays. Clin. Vaccine Immunol. 16, 1405-1412 (2009).
23. Ponnuswamy, N. et al. Oligolysine-based coating protects DNA nanostructures from low-salt denaturation and nuclease degradation. Nat. Commun. 8, 15654-15654 (2017).
24. Koval, M., Preiter, K., Adles, C., Stahl, P. D. & Steinberg, T. H. Size of IgG-Opsonized Particles Determines Macrophage Response during Internalization. Exp. Cell Res. 242, 265-273 (1998).
25. Wang, W. X. et al. Universal, label-free, single-molecule visualization of DNA origami nanodevices across biological samples using origamiFISH. Nat. Nanotechnol. 1-12 (2023) doi:10.1038/s41565-023-01449-5.
26. Thiele, L. et al. Evaluation of particle uptake in human blood monocyte-derived cells in vitro. Does phagocytosis activity of dendritic cells measure up with macrophages? J. Controlled Release 76, 59-71 (2001).
27. Jaumouillé, V. et al. Actin cytoskeleton reorganization by Syk regulates Fcγ receptor responsiveness by increasing its lateral mobility and clustering. Dev. Cell 29, 534-546 (2014).
28. Kern, N., Dong, R., Douglas, S. M., Vale, R. D. & Morrissey, M. A. Tight nanoscale clustering of Fcγ receptors using DNA origami promotes phagocytosis. eLife 10, (2021).
29. Bastings, M. M. C. et al. Modulation of the Cellular Uptake of DNA Origami through Control over Mass and Shape. Nano Lett. 18, 3557-3564 (2018).
30. Galeotti, C., Kaveri, S. V. & Bayry, J. IVIG-mediated effector functions in autoimmune and inflammatory diseases. Int. Immunol. 29, 491-498 (2017).
31. Abdou, N. I. et al. Efficacy of Intravenous Gammaglobulin for Immunoglobulin G Subclass and/or Antibody Deficiency in Adults. Int. Arch. Allergy Immunol. 149, 267-274 (2009).
32. Qureshi, O. S. et al. Multivalent Fcγ-receptor engagement by a hexameric Fc-fusion protein triggers Fcγ-receptor internalisation and modulation of Fcγ-receptor functions. Sci. Rep. 7, 17049 (2017).

Although various embodiments of the disclosure have been described and illustrated, it will be apparent to those skilled in the art in light of the present description that numerous modifications and variations can be made. The scope of the invention is defined more particularly in the appended claims. All referenced documents are incorporated herein by reference.

What is claimed is:

1. An artificial immune complex (IC) free in solution, the artificial IC comprising a nucleic acid (NA) folding comprising stapled NA strands, the NA folding having a surface patterned with addressable sites and epitopes bound to the addressable sites and displayed in three dimensions for recruiting antibodies free in solution.

2. The artificial IC of claim 1, wherein the artificial IC further comprises the antibodies scaffolded to the NA folding such that the fragment antigen-binding (Fab) region of the antibodies are bound to one or more of the epitopes patterned on the NA folding and the Fc portion of the antibodies orients away from the surface of the NA folding, wherein the patterning of the epitopes promotes an immune response against the entire artificial IC.

3. The artificial IC of claim 1, wherein the addressable sites comprise (i) single-stranded NA handles (handles) patterned on the surface of the NA folding, each handle having an end attached to the outer surface of the NA folding, and wherein each epitope includes a single-stranded NA sequence that hybridizes with the handles, or (ii) functional groups and each epitope is bound directly to one functional group on the NA foldings.

4. The artificial IC of claim 1, wherein the epitopes bound to the addressable sites are patterned in regular geometric groupings on the surface of the NA folding, and wherein the epitopes bound to the addressable sites are patterned in pairs, or in clusters of 3 or more copies of epitopes.

5. The artificial IC of claim 4, wherein the NA folding is a 3-dimensional (3D) NA folding, and the regular geometric groupings and epitopes within the regular geometric groupings are radially, axially and azimuthally spaced on the surface of the 3D NA foldings to control antibody binding, structure and/or composition of the artificial IC.

6. The artificial IC of claim 1, wherein the artificial IC comprises a single NA folding in which the epitopes are arranged on the surface in clusters of 2 or more epitopes per cluster, and wherein epitopes within each cluster are spaced apart on the surface of the NA foldings at a distance within a binding tolerance of the antibodies free in solution, and spacing between neighboring clusters of epitopes is outside the binding tolerance of the antibodies free in solution, thereby preventing the antibodies free in solution from binding neighboring clusters of epitopes and from crosslinking epitopes on separate NA foldings.

7. The artificial IC of claim 1, wherein the artificial IC comprises an assembly of multiple NA foldings crosslinked via the fragment antigen-binding (Fab) region of the antibodies, wherein all epitopes patterned on the surface of the NA folding are spaced apart at a distance outside a binding tolerance of the antibodies free in solution, thereby promoting the antibodies free in solution crosslinking between two or more artificial ICs.

8. The artificial IC of claim 1, wherein the artificial IC comprises a mixture of single NA foldings with an assembly of multiple NA foldings, wherein the epitopes are patterned on the surface of each NA folding in clusters of two or more epitopes, and wherein at least one cluster includes at least two epitopes spaced apart at a distance within a binding tolerance of the antibodies free in solution, and at least one cluster includes at least two epitopes spaced apart at a distance that is greater than the binding tolerance of the antibodies free in solution, thereby controlling the number of cross linking antibodies and the overall number of NA foldings in the assembly.

9. The artificial IC of claim 2, wherein the surface of the NA folding is coated with a lysine multimer having a PEG moiety conjugated to the backbone of the lysine multimer and/or to an end of the lysine multimer.

10. The artificial IC of claim 1, wherein the artificial IC comprises an assembly of multiple NA foldings crosslinked via the fragment antigen-binding (Fab) region of the antibodies.

11. The artificial IC of claim 1, wherein the artificial IC comprises a single NA folding.

12. The artificial IC of claim 2, wherein the NA folding carries a cargo, wherein the cargo includes a nucleic acid sequence in the NA folding encoding for a therapeutic or immunomodulatory protein, a small molecule, a macromolecule, an adjuvant peptide, a protein, a chemotherapeutic, and/or an immune-modulatory drug, and wherein the cargo is incorporated into an inner lumen of the NA folding or on an outer surface of the NA folding.

13. A method of manufacturing a synthetic immune-complex (IC), the method comprising:
(a) mixing in an aqueous solution (i) nucleic acid (NA) foldings and epitopes, each NA folding having a surface patterned with addressable sites to bind the epitopes in the solution, or (ii) NA foldings and staple NA strands conjugated with epitopes, thereby obtaining a mixture of epitopes bound to the addressable sites of NA foldings; and
(b) adding antibodies to the mixture of epitopes bound to the NA foldings, wherein the antigen-binding portion of the antibodies binds to the epitope bound to the surface of NA foldings, such that the Fc portion of the antibodies orients away from the surface of the NA folding.

14. The method of claim 13, wherein each addressable site comprises a functional group attached to staple NA strands at specific sites on the surface of the NA folding, and wherein each functional group is an incorporation site for the epitopes.

15. The method of claim 13, wherein each addressable site comprises single-stranded NA handles (handles) patterned at specific sites on the surface of the NA folding, each handle having an end attached to the surface of the NA folding and a free end that orients away from the surface of the NA folding, and each epitopes having a single stranded anti-handle NA sequence, and wherein step (a) includes mixing in the aqueous solution the NA foldings including the handles with the epitopes having the anti-handle NA sequence under conditions favorable for the hybridization of the handles to the anti-handles.

16. The method of claim 13, wherein the epitopes are bound to the addressable sites in clusters of two or more epitopes per cluster, and wherein epitopes within a cluster are space apart at a distance within a binding tolerance of the antibodies free in solution, and spacing between neighboring clusters of epitopes is outside the binding tolerance of the antibodies free in solution thereby preventing the antibodies free in solution from crosslinking epitopes in neighboring pairs within one NA folding and from crosslinking epitopes on separate NA foldings.

17. The method of claim 13, wherein all epitopes patterned on the surface of the NA folding are spaced apart at a distance that is outside a binding tolerance of the antibodies free in solution, thereby promoting the antibodies free in solution crosslinking between two or more artificial ICs.

18. The method of claim 13, wherein the epitopes are bound to the addressable sites in clusters of two or more epitopes per cluster, and wherein each includes (i) epitopes spaced apart within a binding tolerance of the antibodies free in solution, and (ii) epitopes spaced apart on the surface of the NA folding at a distance outside the binding tolerance of the antibodies free in solution, thereby promoting mixture of antibodies free in solution to both cross linking between two or more NA foldings and to binding epitopes within a cluster on a single NA folding.

19. A method of delivering a cargo to a target site in a subject, the method comprising administering to the subject the artificial IC of claim 12, wherein the artificial IC is a multimeric artificial IC comprising an assembly of multiple NA foldings crosslinked via the fragment antigen-binding (Fab) region of the antibodies, or the artificial IC is a monomeric artificial IC comprising a single NA folding, and wherein the target site includes lymph nodes, spleen, tonsils, and/or diseased tissue.

20. A method of inducing an immune response in a subject, the method comprising administering to the subject the artificial IC of claim 2, wherein the artificial IC is a multimeric artificial IC comprising an assembly of multiple NA foldings crosslinked via the fragment antigen-binding (Fab) region of the antibodies, or the artificial IC is a monomeric artificial IC comprising a single NA folding.
